(19) United States
(12) Patent Application Publication
(10) Pub. No.: US 2021/0308326 A1
(43) Pub. Date: Oct. 7, 2021

(54) IMPLANTS TO INDUCE BONE REGENERATION AND USES THEREOF

(71) Applicant: University of lowa Research Foundation, lowa City, IA (US)

(72) Inventors: Aliasger K. Salem, Coralville, IA (US); Behnoush Khorsand Sourkohi, lowa City, IA (US); John E. Femino, Coralville, IA (US); Douglas Fredericks, lowa City, IA (US)

(21) Appl. No.: 17/260,754

(22) PCT Filed: Jul. 18, 2019
(86) PCT No.: PCT/US2019/042445

§ 371 Date: Jan. 15, 2021

Related U.S. Application Data
(60) Provisional application No. 62/699,999, filed on Jul. 18, 2018.

(51) Int. Cl. A61L 27/36; A61L 27/52; A61L 27/54; A61L 27/22; A61L 27/24

(52) U.S. Cl. CPC A61L 27/365; A61L 27/52; A61L 27/54; A61L 27/225; A61L 2400/12; A61L 2300/258; A61L 2300/428; A61L 2300/43; A61L 2430/02; A61L 27/24

(57) ABSTRACT

Disclosed herein are compositions to facilitate bone growth, formation, and/or repair. Methods of using and making the compositions are also disclosed.

### CROSS-REFERENCE TO RELATED APPLICATIONS

This application claims the benefit of the filing date of U.S. application No. 62/699,999, filed on Jul. 18, 2018, the disclosure of which is incorporated by reference herein.

### BACKGROUND

Bone-related conditions are especially difficult to treat in patients with diabetes mellitus (DM). DM is a heterogeneous group of disorders capable of impairing glucose metabolism and is characterized by high concentrations of glucose in the blood (Bell et al., 2001). Type 2 DM (T2DM) is a metabolic disorder resulting from inactivity or obesity and represents about 90-95% of all diagnosed cases of DM globally (Zimmet et al., 2001). It has been projected that by 2025, about 300 million people will be afflicted with DM worldwide (Zimmet et al., 2001). This metabolic disorder has many possible complications, including eventual limb loss when bones fail to heal in the lower leg/ankle/foot (SooHoo et al., 2009; Sohn et al., 2010). It has been documented that there is an association between DM and impaired bone healing (Jiao et al., 2015), decreased bone mineral density (Heap et al., 2004), and increased fracture risk (Okazaki, 2009).

The mechanism by which fracture healing in diabetes is delayed remains poorly understood, however, there is evidence suggesting that osteoblast-like cell proliferation and differentiation is compromised due to high glucose concentrations, thus impacting osteoblast functionality in a diabetic microenvironment. Specifically, it has been demonstrated that osteoblasts differentiate toward adipocytes at the fracture site of T2DM patients, thus hindering the fracture healing process (Hamann et al., 2011; Brown et al., 2014) Furthermore, due to the presence of continuous hyperglycemia, the formation of advanced glycation end products (AGE) is increased, which products are capable of reducing bone quality and bone healing in T2DM patients by signaling through the cell-surface receptor for AGE, RAGE (Cortizo et al., 2003). Previously, it has been shown that there are elevated AGE levels and significantly higher levels of RAGE expressed on osteoblasts during the process of bone healing in T2DM (Furst et al., 2016; Santana et al., 2001). The production of reactive oxygen species, and inflammatory cytokines (such as TNFα) can be induced by AGE binding to RAGE, which can have deleterious effects on bone healing (Brownlee, 2001). Also, it has been found that bone turnover is altered in T2DM with a negative impact on bone formation and bone resorption (Starup-Linde et al., 2016).

### SUMMARY

Described herein are compositions and methods that are useful for inducing bone growth and/or repair. The compositions and methods provide for improved therapeutics to enhance fracture healing and bone regeneration, e.g., such as in bone injuries and in other settings including but not limited to bone fractures or bone degeneration, and in patients with diabetes mellitus (DM). In one embodiment, the compositions are useful to enhance fracture healing and bone regeneration in T2DM patients with poorly controlled blood glucose levels.

As disclosed herein, fracture healing impairment due to DM can be addressed by combining non-viral gene delivery of plasmids independently encoding growth factors, e.g., bone morphogenetic protein-2 (BMP-2) and fibroblast growth factor-2 (FGF-2), which may act synergistically in promoting fracture healing, e.g., as shown in a DM animal model. Both insulin and the hormonally active form of vitamin D3, 1α,25-dihydroxyvitamin D3 (1α,25(OH)₂D₃) (VD3), have been shown to play key roles in regulating bone fracture healing in DM. To investigate if the local delivery of BMP-2 and FGF-2 genes, insulin (INS) and VD3 together could promote bone formation ectopically in Type-2 diabetic rats, a composite having VD3 and insulin containing poly(lactic-co-glycolic acid) (PLGA) microparticles (MPs) embedded in a fibrin gel surrounded by a collagen scaffold impregnated with polyethylenimine (PEI)-(pBMP-2+pFUF-2) nanoplexes was prepared. Using an osteoinduction model, it was demonstrated that local delivery of INS, VD3 and PEI-(pBMP-2+pFGF-2) resulted in a significant improvement in bone generation compared to other treatments. Thus, the combined local release of INS, VD3 and PEI-(pBMP-2+pFGF-2) may be beneficial for promoting bone regeneration in patients with DM. Thus, the composites described herein deliver one or more therapeutics to sites or structures within or on the body of a mammal, e.g., a non-primate mammal such as a canine, feline, bovine, equine, ovine, caprine or swine, or a primate such as a human, which therapeutics may be controllably delivered. e.g., sustained delivery, or delivery before or after delivery of another therapeutic. For example, the composite may control the order of delivery of therapies or the time between the deliveries of therapies. In some embodiments, the delivery of therapy is mediated by a scaffold, gel, and/or alternate delivery vehicle. In some embodiments the timing, location, persistence, rate of release, initial burst release, and/or other characteristics of such delivery is controlled by a scaffold, gel, and/or delivery vehicle. In one embodiment, the composite is useful to stimulate bone regeneration, e.g., in a bone fracture, in diabetic patients.

In one embodiment, the disclosure provides a composition comprising a scaffold loaded with isolated nucleic acid encoding at least two growth factors, e.g., growth factors including but not limited to BMP, FGF, IGF, HGF, PGF, PDGF, TGFB or VEGF; a gel loaded with insulin and at least one bioavailable form of vitamin D, e.g., vitamin D2, vitamin D3 or 25-hydroxyvitamin D (250HD); wherein the scaffold encapsulates or surrounds the gel. In one embodiment, the nucleic acid is linear DNA, plasmid DNA, mRNA, cmRNA, or double-stranded RNA. In one embodiment, the scaffold comprises a natural polymer. In one embodiment, the natural polymer comprises collagen, proteoglycan, alginate, chitosan or extracellular matrix. In one embodiment, the scaffold comprises a synthetic polymer. In one embodiment, the synthetic polymer comprises PLA, PLGA, PLLA or polystyrene. In one embodiment, the bioavailable form of vitamin D comprises calcitriol, cholecalciferol, ergocalciferol, or ercalcitriol. In one embodiment, the gel comprises fibrin. In one embodiment, the insulin is complexed with or encapsulated in a first delivery vehicle. In one embodiment, the first delivery vehicle comprises microparticles or nanoparticles. In one embodiment, the first delivery vehicle comprises cationic or non-cationic polymers, cationic liposomes or cationic emulsions, or a synthetic polymer. In one embodiment, the first delivery vehicle comprises PEI, chitosan, cyclodextrin, dendrimers, PLGA, PLA, or PAMAM. In one embodiment, the plasmid DNA is complexed with or encapsulated in a second delivery vehicle. In one embodiment, the second delivery vehicle comprises a synthetic polymer. In one embodiment, the synthetic polymer comprises PEI, PLGA, PLA, or PAMAM. In one embodiment, the gel controls the initial burst release of the insulin. In one embodiment, the growth factor comprises BMP1, BMP2, BMP3, BMP4, BMP5, BMP6, BMP7, BMP8A, BMP8B, BMP 10, BMP15, FGF1, FGF2, FGF3, FGF4, FGF5, FGF6, FGF7, FGF8, FGF9, FGF10, FGF11, FGF12, FGF13, FGF14, FGF15, FGF16, FGF 17, FGF18, FGF19, FGF20, FGF21, FGF22, FGF23, IGF1, IGF2, HGF, NGF, PDGFA. PDGFB, PDGFC, PDGFD, TGFB1, TGFB@, TGFB3, VEGFA, VEGFB, VEGFC, or VEGFD, or any combination thereof. In one embodiment, the nucleic acid is released from the composition before the bioavailable form of vitamin D. In one embodiment, the bioavailable form of vitamin D is released from the composition before the insulin. In one embodiment, the composition has a cylindrical shape, e.g., a cylindrical shape having a radius of about 1 mm to 20 mm, 5 mm to 15 mm, or 20 mm to 30 mm, and a height of about 1 mm to 10 mm, 5 mm to 15 mm or 5 mm to 10 mm. In one embodiment, the composition has a cylindrical shape having a radius of about 5 mm and a height of 5 mm. In one embodiment, the composition has a spherical shape, e.g., a spherical shape having a radius of about 1 mm to 20 mm, 5 mm to 15 mm, or 20 mm to 30 mm. In one embodiment, the composition has a spherical shape having a radius of about 5 mm. In one embodiment, the composition has a square, rectangular, pyramid, cube, cone, prism or tetrahedron shape. In one embodiment, the composition comprises 0.1 μg to 10 μg, 1 μg to 10 μg, 10 μg to 50 μg, 50 μg to 500 μg, 0.5 mg to 10 mg, 1 mg to 10 mg, 10 mg to 50 mg, or 50 mg to 500 mg of the bioavailable forms of vitamin D. In one embodiment, the composition comprises 0.1 μg to 10 μg, 1 μg to 10 μg, 10 μg to 50 μg, 50 μg to 500 μg, 0.5 mg to 10 mg, 1 mg to 10 mg, 10 mg to 50 mg, or 50 mg to 500 mg of the nucleic acid. In one embodiment, the composition comprises 0.1 μg to 10 μg, 1 μg to 10 μg, 10 μg to 50 μg, 50 μg to 500 μg, 0.5 mg to 10 mg, 1 mg to 10 mg, 10 mg to 50 mg, or 50 mg to 500 mg of insulin.

Further provided is a method to prevent, inhibit or treat bone injury in a mammal, comprising administering the composition disclosed herein to a mammal, e.g., at a site of potential bone injury or a site of bone injury. In one embodiment, the mammal is a human. In one embodiment, the human is suspected to have diabetes mellitus. In one embodiment, the bone injury comprises at least one bone fracture.

### BRIEF DESCRIPTION OF THE DRAWINGS

FIG. 1A-1E. (A) Schematic illustrating a composite scaffold ((INS MPs+VD3)Gel+GAM; (B) Transmission electron micrograph of PEI-(pBMP-2+pFGF-2) nanoplexes. Scale bar=200 nm; (C) TEM micrograph of BMSCs showing the uptake of nanoplexes (blue arrows). Scale bar=1.0 μm; (D) TEM images of the osmotic swelling and rupture of endosomes. Scale bar=1.0 μm (insert scale bar=100 nm); (E1) scanning electron micrographs of INS MPs show spherically shaped MPs with smooth surfaces. Scale bar=40 μm; (E2) SEM micrographs of INS MPs after 28 days of release. Scale bar=20 μm; (E3) Cumulative release profiles of INS from INS MPs and ((INS MPs)Gel) incubated at 37° C. and agitated at 300 rpm in PBS (n=3). Data are presented as mean±SEM.

FIG. 2A-2E. (A) Schematic illustration of the rat surgical procedure, showing lumbar paraspinal and bicep femoris implantation sites; (B) Serum parameters measured in ZDF rats at 0, 7, 14, 21 and 28 days post implantation to assess the potential toxicity of the various composite scaffolds; (C) Blood glucose levels of ZDF rats treated with different implants were monitored over the course of study; (D) ZDF rats weight change over time during treatments. ZDF rats were weighed on days 0, 7, 14, 21, 28 post implantation; (E) ZL rats weight change over time during treatments. ZL rats were weighed on days 0, and 28. Data are presented as mean±SEM.

FIG. 3A-3D. RT-qPCR analysis. Time-course of the expression of genes (A) Runx-2, (B) ALP, (C) OSC and (D) IL1-b involved in osteogenesis in the groups treated with ((INS+VD3)Gel+GAM) implanted scaffolds. Values are expressed as mean±SEM.

FIG. 4A-4D. (A) Representative μCT images (Scale bar=2.5 mm) and (B) 3-dimentional reconstructed, regenerated bone after 28 days of implantation in a ZDF rat intramuscular model. (C) the bone volume and (D) bone surface area of newly formed bone. Statistical analysis was performed using an one way ANOVA followed by Tukey's post-test (***p<0.001, **p<0.01, *p<0.05). Values are expressed as mean±SEM.

FIG. 5A-5E. (A) Schematic illustration of the composite scaffolds ((INS MPs+VD3)Gel+GAM; (B) Transmission electron micrograph of PEI-(pBMP-2+pFGF-2) nanoplexes. Scale bar=200 nm; (C) TEM micrograph of BMSCs showing the uptake of nanoplexes (blue arrows). Scale bar=1.0 μm; (D) TEM images of the osmotic swelling and rupture of endosomes. Scale bar=1.0 μm (insert scale bar=100 nm); (E1) scanning electron micrographs of INS MPs show spherically shaped MPs with smooth surfaces. Scale bar=40 μm; (E2) SEM micrographs of INS MPs after 28 days of release. Scale bar=20 μm; (E3) Cumulative release profiles of INS from INS MPs and ((INS MPs)Gel) incubated at 37° C. and agitated at 300 rpm in PBS (n=3). Data are presented as mean±SEM.

FIG. 6A-6E. (A) Schematic illustration of the rat surgical procedure, showing lumbar paraspinal and bicep femoris implantation sites; (B) Serum parameters measured in ZDF rats at 0, 7, 14, 21 and 28 days post implantation to assess the potential toxicity of the various composite scaffolds; (C) Blood glucose levels of ZDF rats treated with different implants were monitored over the course of study; (D) ZDF rats weight change over time during treatments. ZDF rats were weighed on days 0, 7, 14, 21, 28 post implantation; (E) ZL rats weight change over time during treatments. ZL rats were weighed on days 0, and 28. Data are presented as mean±SEM.

FIG. 7A-7D RT-qPCR analysis. Time-course of the expression of genes (A) Runx-2, (B) ALP. (C) OSC and (D) IL1-b in the groups treated with ((INS+VD3)Gel+GAM) implanted scaffolds. Values are expressed as mean±SEM.

FIG. 8. Differential expression (DE) of mRNAs relative to (Gel+CM) in logarithmic scale (Log 2 scale) are illustrated as a heat map. A fold change ≥4 relative to (Gel+CM) was used as a filter to identify the DE genes.

FIG. 9A-9D. (A) Representative μCT images (Scale bar=2.5 mm) and (B) 3-dimentional reconstructed, induced bone after 28 days of implantation in a ZDF rat intramuscular model. (C) the bone volume and (D) bone surface area of newly formed bone. Statistical analysis was performed using an one way ANOVA followed by Tukey's post-test (***p<0.001, **p<0.01, *p<0.05). Values are expressed as mean±SEM.

FIG. 10A-10B. Histological evaluation of bone formation at 4 weeks (A) Representative histology images (Scale bar=200 μm) of induced bone after 28 days of implantation in a ZDF rat intramuscular model. (B) The new bone area formed after 28 days of implantation of indicated treatments in ZDF and ZL rats. Values are expressed as mean±SEM. (B: Bone; I: Implant; and M: Muscles)

### DETAILED DESCRIPTION

### General Terminology

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment.” “nucleic acid sequence or segment,” or “polynucleotide” can also be used interchangeably with gene, cDNA, DNA and RNA encoded by a gene, e.g., genomic DNA, and even synthetic DNA sequences. The term also includes sequences that include any of the known base analogs of DNA and RNA.

By “fragment” or “portion” is meant a full length or less than full length of the nucleotide sequence.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous)nucleotide sequence.

As used herein, “sequence identity” or “identity” in the context of two nucleic acid sequences makes reference to a specified percentage of residues in the two sequences that are the same when aligned by sequence comparison algorithms or by visual inspection.

As used herein, “percentage of sequence identity” means the value determined by comparing two optimally aligned sequences, wherein the portion of the polynucleotide sequence may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters.

“Treating” as used herein refers to ameliorating at least one symptom of, curing and/or preventing the development of a disease or a condition.

The terms “protein,” “peptide” and “polypeptide” are used interchangeably herein.

The term “molecule” refers to an atom or atoms held together by chemical bonds, including chemical compounds. Chemical bonds include covalent bonds, ionic bonds, metallic bonds, and coordinate covalent bonds.

Examples of a molecule include but are not limited to a protein, DNA. RNA, nucleic acids, and certain fluorescent dyes.

The terms “bind” or “bound” refer to two or more molecules that are in contact through various types of non-covalent interactions that do not involve the sharing of electrons, but rather involve more dispersed variations of electromagnetic interactions between molecules or within a molecule. Examples of such interactions are electrostatic interactions (e.g. ionic bonds, hydrogen bonds and halogen binding), Van der Waals forces (e.g. dipole-dipole, dipole-induced dipole and London dispersion forces), π-effects (e.g. π-π interactions, cation-π and anion-π interactions, and polar-π interactions), and the hydrophobic effect.

The term “complex” as a noun refers to two or more molecules that are bound to each other, e.g. protein-protein, protein-DNA, protein-RNA, and/or polymer-nucleic acid complexes. The term “complex” as a verb refers to the formation of a complex or presence of a complex.

The term “mixture” refers to a collection of things, e.g. molecules, of one or more types.

The term “growth factor” refers to the genes listed in Table 2 and the protein products of those genes. It can also refer to various types of nucleic acid encoding the protein products of those genes. Those various types of nucleic acid include but are not limited to plasmid DNA, linear DNA, mRNA, double stranded RNA, and cmRNA.

Linear DNA is single or double stranded DNA that is not circularized.

Plasmid DNA is the standard type of plasmid DNA used in laboratories and equivalents of such plasmid DNA.

mRNA is messenger RNA and its equivalents.

cmRNA is chemically modified RNA. Chemically modified RNA is RNA that is produced from nucleotides other than guanine, uracil, adenine, and cytosine. For example, cmRNA can be produced with 5-methylcytosine and/or pseudouridine, cmRNA can also be produced with many other nucleotides. Many other molecules can be used to produce cmRNA.

Double stranded RNA is one or more molecules of RNA that contain molecular bonds between complementary sequences.

The term “scaffold” refers to linked polymer chains that provide physical support, physical stability or reduce movability to molecules. The links of a scaffold can be physical or chemical and can be covalent, ionic or non-ionic or other types of links. Scaffolds can form substantially two-dimensional or three-dimensional net-like or mesh-like structures. Scaffolds can be formed with various geometries. For example, components of scaffolds can form substantially triangular, substantially quadrilateral, or irregular shapes. More examples of such shapes can be substantially similar to tetrahedron, pyramid, hexahedron, other polyhedra, or irregular three-dimensional shapes. There are many other shapes that can be formed by scaffold components. Scaffolds can be produced comprising natural and/or synthetic polymers.

The term “gel” refers to a cross-linked system of polymers that form a three-dimensional network and contains liquid. Cross-links can be physical or chemical and can be covalent, ionic or non-ionic or other types of cross-links. A gel can be jelly-like or gelatinous. Gels can be produced using natural and/or synthetic polymers.

The term “delivery vehicle” refers to a composition that facilitates the delivery of a therapy to its target location by complexing directly or indirectly with the therapy and/or carrying the therapy. Delivery vehicles can facilitate the movement of therapy through the body, tissue, joints, cells, organelles, membrane-bound or membraneless regions of cells, or other structures of a patient, human, mammal, or organism.

The term “encapsulates” refers to the state wherein one structure substantially completely encloses another structure. For example, a scaffold can completely enclose, or encapsulate, a gel.

The term “surround” refers to the state wherein one structure covers much of the surface of another structure. For example, a scaffold can cover most of the surface of a gel. For example, the surrounding structure can cover the rectangular surface of a cylindrical surrounded structure and leave one or both of the circular faces of the cylindrical surrounded structure exposed. One structure can surround another structure by covering more than 50%, 60%, 70%, 80%, 90%, 95%, 98%, 99%, or more of the surrounded structure.

The term “initial burst release” refers to the initial rapid release of a molecule or molecules from a structure during the burst phase. Such structures include but are not limited to one or more scaffolds, gels, delivery vehicles, microparticles, nanoparticles, and/or other structures. A burst phase occurs when a molecule or molecules is initially rapidly released from, diffuses from and/or otherwise exits a structure or structures. The burst phase can then be followed by slower release, diffusion, or exit from the structure or structures. For example, release from such a structure can occur according to biphasic Michaelis-Menten kinetics, whereby an initial burst phase is followed by a steady release rate.

PLA is polylactic acid.

PLGA is poly(lactic-co-gly colic) acid.

PLLA is poly-_{L}-lactic acid.

PEI is polyethylenimine.

### INS, VD3 and Growth Factors

The growth, division, differentiation, degradation, and/or other characteristics of cells, tissue, organs, joints, and/or other bodily structures can be regulated by molecules. Such molecules include vitamins, hormones, peptides, and/or other molecules. More than one type of molecule can regulate the characteristics of bodily structures cooperatively or antagonistically and can do so by acting in sequence. Growth factors fulfill functions including but not limited to the growth, division, and differentiation of cells. Growth factors can be any of many structures, such as vitamins, hormone, small molecules, peptides, and/or other forms. Sometimes growth factors can be used interchangeably and that sometimes growth factors cannot be used interchangeably. Growth factors can be used as therapy for disease, injury, and/or other conditions and disorders. Appropriate combinations of growth factors can be used as therapy to treat bone injuries, such as bone fracture. Appropriate combinations of growth factors, as disclosed herein, can be used to treat bone injuries in patients with diseases, conditions, and/or disorders, such as diabetes mellitus. In some embodiments there is a growth factor or growth factors. In some embodiments the patient is suspected to have diabetes mellitus.

Increased bone regeneration was observed by combining non-viral gene delivery of plasmids independently encoding BMP-2 and FGF-2, resulting in a synergistic effect on the promotion of fracture healing in a chronic diabetic animal model (Khorsand et al., 2017). BMP-2 and FGF-2 are participants in the process of bone healing and regeneration, being capable of synergistically enhancing osteoblast recruitment and proliferation as well as stimulating angiogenesis (Xiong et al., 2017; Khorsand et al., 2017).

INS is an anabolic agent for bone (Thrailkill et al., 2005) and its effect on fracture healing in DM and healthy animals has been documented (Hough et al., 1981; Beam et al., 2002). Several studies have indicated that INS acts directly on the callus to reverse impaired boney healing and positively regulate bone fracture healing at the systemic (Beam et al., 2002) and local levels (Gandhi et al., 2005). INS possibly acts by enhancing bone formation and decreasing bone resorption (Bean et al., 2002). A previous study has demonstrated that local INS therapy could stimulate fracture site osteoblast proliferation, collagen production, alkaline phosphatase production and new bone content (mineralization) in diabetics (Jianhong et al., 2010; Pun et al., 1989). Also, it has been shown that local INS treatment at the fracture site can accelerate fracture healing in animals (Paglia et al., 2013; Park et al., 2013).

There is an association between insufficiency of VD3 and the incidence of DM (Maxwell et al., 2011) and that high serum levels of VD3 can reduce the risk of DM significantly (Parker et al, 201). VD3 (Plum et al., 2010), can directly bind to the VD3 receptor to prevent oxidative stress and upregulate glucose metabolism (Gradinaru et al. 2012; Manna et al., 2017). Yet the molecular mechanism by which VD3 stimulates glucose homeostasis is not clear. Furthermore, many animal models support the positive correlation between bone healing and systemic VD3 supplementation, in which the VD3 metabolites promote bone remodeling and improve osseointegration of the implants in diabetic animals (Wu et al., 2013; Xiong et al., 2017). Nevertheless, studies exploring the distinct effect of local VD3 treatment on bone healing in diabetic animals are rare.

The delivery of INS and VD3 to the fracture site may normalize cellular proliferation, chondrogenesis, mineralization, cartilage content and biomechanical properties of the fracture callus without affecting systemic blood glucose parameters.

As described herein, an unique composite material was developed to enhance the synergistic effects of BMP-2 and FGF-2 gene delivery to fractures in animals with DM. Local delivery of INS and VD3 along with non-viral gene delivery of BMP-2 and FGF-2 significantly ameliorated impaired diabetic fracture healing, as evidenced by enhanced bone regeneration and improved osseointegration into a surgically implanted sub-muscular device. The osteogenic capability of the composite material was found to promote bone formation after ectopic implantation in Zucker diabetic fatty (ZDF) and Zucker lean (ZL) rats.

In particular, to provide for sustained-release delivery of the INS, PLGA MPs encapsulating INS (INS MPs) were prepared. Delivery of INS directly to the implant site from INS MPs can improve osseointegration after implantation in diabetic rats (Wang et al., 2011). A bilayer scaffold composed of collagen (outer layer) and fibrin gel (inner layer) was prepared where INS MPs and VD3 were incorporated into the fibrin gel to achieve a controlled release of VD3 and a more sustained release of the INS compared to the INS MPs alone. This gel was then surrounded by a collagen scaffold harboring PEI-(pBMP-2+pFGF-2) nanoplexes. The bone generative capability of this composite when implanted intramuscularly into the ZDF and ZL rats was demonstrated compared to the control groups.

### EXEMPLARY EMBODIMENTS

In one embodiment, a composition to treat bone injury is provided. The composition includes a scaffold loaded with nucleic acid encoding at least two growth factors, e.g., one or more selected from the list in Table 2; a gel loaded with insulin and at least one form of bioavailable form of vitamin D, wherein the scaffold encapsulates or surrounds the gel. In one embodiment, the nucleic acid is linear DNA, plasmid DNA, mRNA, cmRNA, or double-stranded RNA. In one embodiment, the composition further comprises a nucleic acid molecule that encodes at least two growth factors, e.g., from those listed in Table 2. In one embodiment, the scaffold comprises a natural polymer, for example, comprising collagen, proteoglycan, alginate, chitosan or extracellular matrix. In one embodiment, the scaffold comprises a synthetic polymer, e.g., comprising PLA, PLGA, PLLA or polystyrene. In one embodiment, the bioavailable form of vitamin D comprises calcitriol. In one embodiment, the bioavailable form of vitamin D comprises ercalcitriol. In one embodiment, the gel comprises fibrin. In one embodiment, the composition further comprises insulin complexed with a first delivery vehicle. In one embodiment, the first delivery vehicle comprises microparticles or nanoparticles. In one embodiment, the first delivery vehicle comprises cationic or non-cationic polymers. In one embodiment, the first delivery vehicle comprises PEI, chitosan, cyclodextrin or dendrimers. In one embodiment, the first delivery vehicle comprises cationic liposomes or cationic emulsions. In one embodiment, the first delivery vehicle comprises a synthetic polymer, e.g., a synthetic polymer comprising PEI, PLGA, PLA, or PAMAM. In one embodiment, the PEI comprises branched PEI. In one embodiment, the plasmid DNA is complexed with a second delivery vehicle. In one embodiment, the second delivery vehicle comprises a synthetic polymer. In one embodiment, the synthetic polymer in the second delivery vehicle comprises PEI, PLGA, PLA, or PAMAM, e.g., the PEI comprises branched PEI. In one embodiment. BMP-2 is one of the growth factors. In one embodiment, FGF-2 is one of the growth factors. In one embodiment, BMP-2 and FGF-2 are the growth factors. In one embodiment, the gel controls the initial burst release of the insulin. In one embodiment, the nucleic acid is released over hours to days. In one embodiment, the bioavailable form of vitamin D is released over hours to days. In one embodiment, the insulin is released over days to weeks. In one embodiment, the nucleic acid is released before the bioavailable form of vitamin D. In one embodiment, the nucleic acid is released before the insulin. In one embodiment, the bioavailable form of vitamin D is released before the insulin.

Also provided is a method of using the composition, e.g., to treat bone injury by administering the composition to a patient at a site of bone injury in the patient. In one embodiment, the nucleic acid in the composition is linear DNA, plasmid DNA, mRNA, cmRNA, or double-stranded RNA. In one embodiment, the nucleic acid molecule encodes at least two growth factors. In one embodiment, the scaffold comprises a natural polymer such as collagen, proteoglycan, alginate, chitosan or extracellular matrix. In one embodiment, the scaffold comprises a synthetic polymer such as one comprising PLA, PLGA, PLLA or polystyrene. In one embodiment, the bioavailable form of vitamin D comprises calcitriol. In one embodiment, the bioavailable form of vitamin D comprises ercalcitriol. In one embodiment, the gel comprises fibrin. In one embodiment, the composition further comprises insulin complexed with a first delivery vehicle. In one embodiment, the first delivery vehicle comprises microparticles or nanoparticles. In one embodiment, the first delivery vehicle comprises cationic or non-cationic polymers. In one embodiment, the first delivery vehicle comprises PEI, chitosan, cyclodextrin or dendrimers. In one embodiment, the first delivery vehicle comprises cationic liposomes or cationic emulsions. In one embodiment, the first delivery vehicle comprises a synthetic polymer such as one comprising PEI, PLGA, PLA, or PAMAM. In one embodiment, the PEI comprises branched PEI. In one embodiment, the plasmid DNA is complexed with a second delivery vehicle. In one embodiment, the second delivery vehicle comprises a synthetic polymer. In one embodiment, the synthetic polymer comprises PEI, e.g., branched PEI, PLGA. PLA, or PAMAM. In one embodiment, BMP-2 is one of the growth factors. In one embodiment, FGF-2 is one of the growth factors. In one embodiment, BMP-2 and FGF-2 are the growth factors. In one embodiment, the gel controls the initial burst release of the insulin. In one embodiment, the patient is suspected to have diabetes mellitus. In one embodiment, the bone injury comprises at least one bone fracture. In one embodiment, the patient is suspected to have diabetes mellitus and the bone injury comprises at least one bone fracture. In one embodiment, the patient is suspected to have diabetes mellitus and the bone injury is a bone fracture.

The invention will be further described by the following non-limiting examples

### Example 1

### Materials and Methods

### Materials:

Resomer® % RG503 (PLGA 50:50, IV 0.32-0.44 dL/g) was obtained from Boehringer Ingelheim Pharma Gmbh & Co (Ridgefield, Conn.). Poly(vinyl alcohol) (PVA; Mowiol® 8-88) was purchased from Sigma-Aldrich® (St. Louis, Mo.). Insulin from bovine pancreas powder, and Cholecalciferol (activated VD3) were acquired from Sigma-Aldrich. Branched PEI (mol. wt. 25 kDa) and the GenElute™ HP endotoxin-free plasmid maxiprep kit were purchased from Sigma-Aldrich. Plasmid DNA (6.9 Kb) encoding BMP-2 protein and plasmid DNA (4.9 Kb) encoding FGF-2 were purchased from Origene Technologies, Inc. (Rockville, Md.). Absorbable type-I bovine collagen was purchased from Zimmer Dental Inc. (Carlsbad, Calif.). TISSEEL™ Fibrin Sealant was obtained from Baxter Healthcare Corp (Deerfield, Ill.). The RNeasy Mini Kit was purchased from Qiagen Inc (Germantown, Md.). The TaqMan Reverse Transcription Reagents and 18S-rRNA were purchased from Applied Biosystems (Foster City, Calif.). All primers were obtained from Integrated DNA Technologies (Coralville, Iowa). Micro BCA™ Protein Assay Kit and RNAlater™ Stabilization Solution was obtained from Thermo Scientific (Pittsburgh, Pa.). Human bone marrow stromal cells (BMSCs) were purchased from the American Type Culture Collection (ATCC®, Manassas, Va.). Dulbecco's Modified Eagle's Medium (DMEM), trypsin-EDTA (0.25%, 1× solution) and Dulbecco's phosphate buffered saline (PBS) were purchased from Gibco® (Invitrogen™, Grand Island, N.Y.). Fetal bovine serum (FBS) was obtained from Atlanta Biologicals® (Lawrenceville, Ga.). Gentamycin sulfate (50 mg/ml) was purchased from Mediatech Inc. (Manassas, Va.). All other chemicals and solvents used were of reagent grade from Sigma Aldrich.

### Bone Marrow Stromal Cells (BMSCs) Culture:

BMSCs were cultured and maintained in DMEM (supplemented with 10% FBS, 1 mM Glutamax™ (Gibco), 1 mM sodium pyruvate (Gibco), and 1% gentamycin (50 μg/ml)) in a humidified incubator at 37° C. and 5% CO₂ flow (Sanyo Scientific Auto flow, Infrared direct heat CO₂ incubator). BMSCs were passaged using 0.25% trypsin-EDTA (Invitrogen™). In this study, BMSCs were used at passages 3 to 4. Cells were cultured on 75 cm² polystyrene cell culture flasks (Corning, N.Y., USA). The BMSCs were mycoplasma-free as determined by a MycoAlert mycoplasma detection kit (Lonza).

### Composite Design and Fabrication:

Isolation of Plasmid DNA (pDNA) Encoding BMP-2, and FGF-2 and Fabrication of PEI-pDNA Nanoplexes:

To amplify the plasmid, the pDNAs encoding BMP-2, and FGF-2 were independently transformed into chemically competent E. coli DH5α™. Subsequently pDNAs were extracted, purified and analyzed for purity as described previously (Alturi et al., 2015). Then, PEI-pDNA nanoplexes (200 μL) containing 25 μg of pFGF-2 and 25 μg of pBMP-2 were fabricated at a molar ratio of PEI amine (N) to pDNA phosphate (P) groups of 10 as described previously (Khorsand et al., 2017).

Preparation of Gene Activated Matrixes (GAMs):

Absorbable type-I bovine collagen was cut into cylindrical scaffolds (radius=5 mm; height=5 mm), then a 4 mm sterile biopsy punch was used to remove the central core of the scaffold, yielding a ring-shaped construct. Afterwards, the PEI-(pBMP-2+pFGF-2) nanoplex solution (200 μL) was injected into the collagen scaffolds using a sterile 28 gage needle and then the GAMs were frozen at −20° C. until required. Collagen matrices (CM) injected with 200 μL of sterile water (RNase and DNase free) were used as controls.

Fabrication of INS containing PLGA MPs (INS MPs):

INS MPs were prepared using the water-in-oil-in-water (W/O/W) double emulsion method. Briefly, 12.5 mg of lyophilized INS powder was dissolved in 200 μL 0.01 N HCl, and the pH adjusted to 4.0. Aqueous INS solution was mixed with 1.5 mL of dichloromethane (DCM) containing 200 mg of PLGA, then sonicated at an energy output level of 40% amplitude for 30 s. The primary emulsion was then re-emulsified with 30 mL of 1% PVA aqueous solution using a homogenizer at 6500 rpm for 30 s. The W/O/W emulsion was stirred for 1.5 h at room temperature, allowing the DCM to evaporate. The emulsion gradually solidified as the solvent diffused from the emulsion droplets into the external phase. The resulting MPs were collected by centrifugation using at 29×g for 5 min, resuspended in 30 mL of Nanopure sterile water, and washed twice with Nanopure sterile water (Thermo Scientific™ Nanopure™). Particles were then suspended in 5 mL of Nanopure sterile water which was frozen at −20° C. for 4 h and lyophilized for 18 hours at collector temperature of −53° C. and 0.08 mBar pressure using a FreeZone 4.5-L Benchtop Freeze Dry System (Labconco Corporation). In this study, INS treatments were provided as INS encapsulated in MPs which will be referred as INS MPs for simplification and the control blank PLGA particles will be referred to as bMPs.

Preparation of Fibrin Gel Loaded with INS MPs and VD3:

The fibrin sealant TISSEEL kit was used, which is composed of sealer protein solution (100 mg/mL fibrinogen) and thrombin solution (500 units/mL thrombin). Fibrin gels loaded with INS MPs and VD3 were prepared by diluting fibrinogen solution in HBSS buffer to obtain 12.5 mg/mL fibrinogen solution. Then 11 mg of INS MPs (10 units, equivalent of 0.455 mg of INS) and 5 μg of the active form of VD3 (calcitriol; 1α, 25(OH)₂D₃) were added to the thrombin solution to form a suspension containing 10 units/mL of thrombin. Finally, the two components were mixed simultaneously at a 1:1 ratio, forming a fibrin clot on delivery. Either in control or treatment groups fibrin gel was prepared as described and was incorporated into the GAM core.

Fabrication of Final Composite (GAMs Loaded with INS MPs and VD3 Gel):

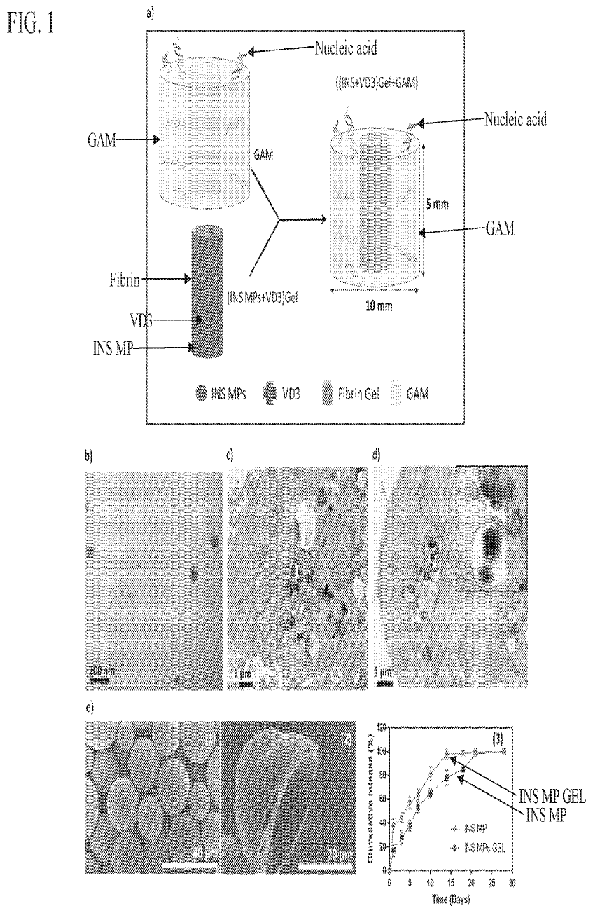

The final construct was prepared by injection of fibrin clot into the ring-shaped GAMs (collagen scaffold containing nanoplexes of pDNA (pBMP-2+pFGF-2)). Then the implants were frozen at −20° C. until required (FIG. 1a).

### Size and Zeta Potential Measurements

Using a Zetasizer Nano ZS particle analyzer, via dynamic light scattering (DLS) technique, particle size and zeta potential of nanoplexes were measured (Malvern Instrument Ltd., Southborough, Mass.). Using an aqueous solution of the nanoplexes the size was measured at 1730 backscatter detection in disposable polystyrene cuvettes and zeta potential was measured in a zeta potential folded capillary cell at 25° C.

### Microscopic Evaluation of the MPs and Nanoplexes

Scanning Electron Microscopy (SEM):

The surface morphology of the INS MPs was performed after fabrication and also at the end of the release study using scanning electron microscopy (SEM, Hitachi S-4000, Schaumburg, Ill.). Briefly, 0.05 mg/mL INS MPs were added onto silicon wafers and air-dried for 24 h and the wafers were then placed on adhesive carbon tabs mounted on SEM specimen stubs. All the specimen stubs were sputter-coated with approximately nm of gold/palladium by ion beam evaporation (argon-beam K550 sputter coater (Emitech Ltd). Images were captured using the SEM operated at 5 kV accelerating voltage (5-4800, Hitachi High-Technologies).

Transmission Electron Microscopy (TEM):

The shape of PEI-(pBMP-2+pFGF-2) nanoplexes prepared at N/P ratio of 10 as well as nanoplex uptake by BMSCs were visualized by transmission electron microscopy (TEM, JEOL JEM-1230) equipped with a Gatan UltraScan 1000 2 k×2 k CCD acquisition system (JEOL USA Inc.). In short, 10 μL of the PEI-pDNA nanoplexes (containing of 25 μg of pFGF-2 and 25 μg of pBMP-2) was absorbed onto carbon-coated grids for 30 s (400-mesh TEM carbon grid by Auto 306 BOC Edwards). All of the TEM grids were pre-coated with a Formvar solution (0.5%) in an ethylene dichloride film ((Electron Microscopy Sciences (EMS)). Then, using Whatman filter paper, the excess sample liquid was removed and air dried.

Using TEM the cellular uptake of nanoplexes was examined. BMSCs were seeded at 10⁵ cells/well into 12 well plates for 24 h. BMSCs then were incubated with 20 μl (1 μg pDNA) of complexes (N/P ratio of 10) for 4 h in the presence of serum-free medium. Then the serum-free medium was replaced with growth medium containing serum. At 48 h post transfection, BMSCs were fixed for 30 min with glutaraldehyde (2.5%) in a sodium cacodylate buffer (0.1 M, pH 7.4, (EMS)). Afterwards, cells were rinsed twice for 4 min each with cacodylate buffer (0.1 M, pH 7.4, (EMS)). Then to improve the efficacy of the fixation and escalate the electron density, BMSCs were treated with 1% osmium tetroxide (EMS) for 30 min. The fixed BMSCs were then stained with uranyl acetate (2.5%, (EMS)) for 5 min, following a double wash with distilled water. Samples were then dehydrated gradually by sequentially incubating in 25%, 50%, 75% and 95% ethanol for 4 min per solution, followed by two final 5 min incubations with 100% ethanol. Finally, to embed the dehydrated samples in Epon (Ted Pella Inc.), samples were infiltrated with a mixture (1:1) of ethanol:Epon for 30 min, and then fixed in Epon for 8 h at 70° C. Using a Leica EM UC6 Ultramicrotome MZ6 (Reichert Technologies), thin sections of samples were prepared (50-70 nm). Finally, these sections were mounted on a Formvar-coated 400-mesh TEM carbon grid, and the images were obtained using TEM, JEOL JEM-1230.

### Quantification of INS Loading and Encapsulation Efficiency of INS MPs:

To quantify INS loading, INS MPs (7.1 mg) was dissolved in chloroform (1 mL), and then mixed with 0.01 N HCL (2 mL) and shaken vigorously to allow for the active ingredient to migrate to the aqueous phase for at least 30 min. The aqueous phase (top layer) was collected and neutralized with 1N NaOH (pH 7.0). Then drug loading (DL) and encapsulation efficiency (EE) were determined using the Micro BCA™ Protein Assay Kit following the manufacturer's recommended protocol (Thermo Scientific). DL and EE were calculated according to the following equations.

$$Drug Loading ( \frac{drug ( µg )}{MPs ( mg )} ) = \frac{Weight of insulin entrapped within MPs ( µg )}{Total weight of MPs ( mg )}$$

$$Encapsulation Efficiency ( \% ) = \frac{Weight of insulin entrapped within MPs ( mg )}{Total weight of initial insulin ( mg )} \times 100$$

### In Vitro Kinetics of Insulin Release:

Insulin release directly from INS MPs and from fibrin gel containing INS MPs was investigated using Micro BCA™ Protein Assay. For assessment of the INS release from INS MPs and gel approximately 10.5 mg of INS MPs was added in 1 mL of PBS (1×) into a 2 mL micro-centrifuge tube or by completely submerging the fibrin gel (100 μL) containing INS MPs (10.5 mg) in 1 mL of PBS (1×) into scintillation vial. The samples were incubated under shaking (300 rpm) at 37° C. For the direct release of INS from INS MPs at regular time intervals, micro-centrifuge tubes were centrifuged at 180×g for 8 min, supernatant was harvested for further analysis and INS MPs were resuspended in fresh PBS. To quantify the INS release from fibrin gels containing INS MPs, at regular time intervals supernatants were taken and replaced with fresh PBS. And the amount of released INS was estimated using the Micro BCA™ Protein Assay Kit according to the manufacturing protocol. All samples were analyzed in triplicate and stored at −20° C. until further analysis.

### Animal Models and Surgical Plan:

The study was approved by, and conducted according to guidelines established by, the University of Iowa Institutional Animal Care and Use Committee (IACUC), Iowa. Adult male Zucker diabetic fatty (ZDF) weight 0.34 kg and Zucker lean (ZL) weight 0.3 kg rats at ages 8-10 weeks were purchased from Charles River Laboratories (Wilmington, Mass.) and housed and cared for in the animal facilities. The bone forming capacity of the implants were studied in an intramuscular implantation site using ZDF and ZL rats. The rats were maintained on a heating pad (37° C.) and were anesthetized by continuous isoflurane inhalation through a vaporizer (0.5-5.0%) prior to implantation and the surgical sites were shaved and disinfected with a 30% betadine solution.

Lumbar Paraspinal Sites:

A skin incision was made approximately 1 cm off of midline on one side of the lumbar spine and bupivacaine (0.5%) was dripped onto the muscle prior to dissection. The paraspinous muscle was exposed and an incision, approximately 1.5 cm in length, was made through the fascia and the underlying muscles separated to create a pocket for implantation. The procedure was repeated on the contralateral side of the spine. Soft tissue and skin was closed in layers using absorbable suture material.

Bicep Femoris Sites:

An approximately 0.5-1.5 cm skin incision was made over the biceps femoris muscle on both limbs and bupivacaine (0.5%) was dripped on the muscle prior to dissection. Pockets were made in each biceps femoris by blunt and sharp dissection, parallel to the muscle fiber long axis. The muscles and skin incision were closed using absorbable suture material.

### Experimental Design:

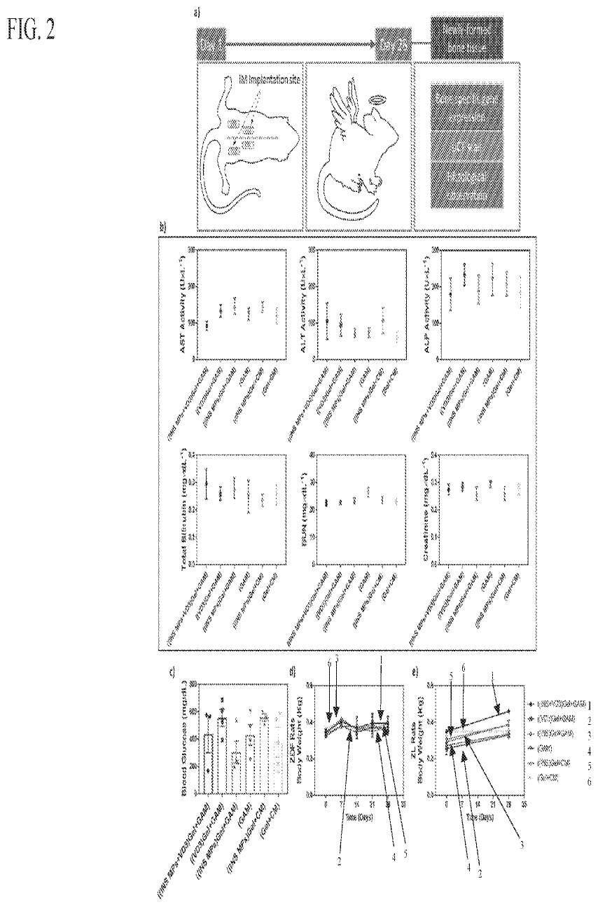

Scaffolds were implanted (four implants per rat) into intramuscular (IM) pockets in the hind limb and back site of the animals. One in each biceps femoris muscle (right and left leg), and one in each dorsal paraspinous muscle (right and left side) (FIG. 2a). Animals were randomly assigned to the six following treatment groups: 1) Implant 1: ((INS MPs+VD3)Gel+GAM), (n=40); 2) Implant 2: ((VD3)Gel+GAM), (n=40); 3) Implant 3: ((INS MPs)Gel+GAM), (n=40); 4) Implant 4: GAM, (n=40); 5) Implant 5: ((INS MPs)Gel+CM), (n=40): and 6) Implant 6: (Gel+CM), (n=40). Animals were monitored twice daily for 5 days during postoperative recovery for any clinical signs of illness, fracture, or reaction to treatment. At 14, 21, and 28 days after surgery, animals were euthanized by C02 inhalation, and the implantation sites with surrounding bone were removed and collected for subsequent analysis.

### Weight and Blood Sample Analysis:

To evaluate glycemia and monitor the potential toxicity associated with the scaffolds, the weights of individual rats were monitored and recorded at 7, 14, 21, and 28 days post-surgery. In addition, blood samples were collected from the tail prior to treatment and at the end of the treatment regimen (7, 14, 21, and 28 days after surgery). The blood serum was separated by centrifugation (1000×g at 4° C. for 10 min) and stored at −80° C. Serum samples were shipped to IDEXX Laboratories (Sacramento, Calif.) for toxicity analyses.

### RNA Extraction and Quantitative Real Time Polymerase Chain Reaction (O-PCR):

On days 14, 21, and 28 scaffolds were explanted and stored in RNAlater™ Stabilization Solution. Scaffolds were submerged in 5 volumes of RNAlater solution (1 g tissue required 5 mL of solution), and stored at 4° C. overnight. The supernatant was then removed and samples were stored at −80° C. until ready for use. Frozen implants were placed in an RNase-free mortar and pestle and ground into a powder while immersed in liquid nitrogen. Then the total RNA was extracted using the RNeasy Mini Kit (Qiagen) according to the manufacturer's instructions. Purified RNA was then reversely transcribed with random hexamers using High-Capacity cDNA Reverse Transcription kit (Applied Biosystems) in the thermocycle system (Bio-Rad). The expression levels of genes involved in osteogenesis were then investigated using the TaqMan Universal PCR Master Mix on QuantStudio 3 Real-Time PCR System. Quantitative PCR was carried out using the primers and probes listed in Table 1 with hypoxanthine guanine phosphoribosyl transferase (HRPT) and ubiquitin C (UBC) as the internal controls. Each 20 μL PCR reaction well contained 2 μL of cDNA, 1 μL of primer-probe mix, and 10 μL of 2× PrimeTime® Gene Expression master mix with a ROX passive reference dye. Cycling conditions were 50° C. for 2 min, and 90° C. for 3 min followed by 40 cycles of 90° C. for 15 s (denaturation) and 60° C. for 1 min (annealing and extension). Analysis of data was performed using the auto-threshold baseline and the 2c method. The expression levels of the target genes were normalized to the expression level of the house keeping genes. Each sample was run in duplicate and values represent the mean of at least 2 replicates.

TABLE 1
Probe and primer sequences.

| | Probe | Forward | Reverse |
|---|---|---|---|
| Runx-2 | TGA AAC TCT | GCC AGG | CGT CCA CTG TCA |
| | TGC CTC GTC | TTC AAC | CTT TAA TAG CTC |
| | CGC TC (SEQ | GAT CTG | (SEQ ID NO: 3) |
| | ID NO: 1) | A (SEQ | |
| | | ID NO: | |
| | | 2) | |
| OSC | CCA GCA GAG | AGA CCT | GCT TGG ACA TGA |
| | TGA GCA GAG | AGC AGA | AGG CTT TG (SEQ |
| | AGA GG (SEQ | CAC CAT | ID NO: 6) |
| | ID NO: 4) | GA (SEQ | |
| | | ID NO: | |
| | | 5) | |
| ALP | TCT GGA ACC | AAA CCT | TCC GAT TCA ACT |
| | GCA CTG AAC | AGA CAC | CAT ACT GCA T |
| | TGC T (SEQ | AAG CAC | (SEQ ID NO: 9) |
| | ID NO: 7) | TCC (SEQ | |
| | | ID NO: | |
| | | 8) | |
| ILl-b | TGG CIT ATG | GTG CIG | TTG TCG TTG CTT |
| | TTC TGT CCA | TCT GAC | GTC TCT CC (SEQ |
| | TTG AGG TGG | CCAT GT | ID NO: 12) |
| | (SEQ ID NO: | (SEQ ID | |
| | 10) | NO: 11) | |
| HRPT | TGG ATA CAG | GGT GAA | GCT TTT CCA CTT |
| | GCC AGA CTT | AAG GAC | TCG CTG ATG |
| | TGT TGG ATT | CTC TCG | (SEQ ID NO: 15) |
| | (SEQ ID NO: | AAG (SEQ | |
| | 13) | ID NO: | |
| | | 14) | |
| UBC | CCC AAG AAC | GAC AGG | AAA ACT AAG ACA |
| | AAG CAC AAG | CAA GAC | CCT CCC CAT C |
| | AAG GGC (SEQ | CAT CAC | (SEQ ID NO: 18) |
| | ID NO: 16) | TC (SEQ | |
| | | ID NO: | |
| | | 17) | |

### Micro-Computed Tomography:

The three-dimensional x-ray micro-computed tomography (ACT) imaging was performed to quantitate the ectopic bone formation in the presence of various treatments. High resolution Skyscan 1176 (Kontich, Belgium) was used with the following settings: voltage 50 KeV, current 500 μA, exposure 1050 ms and slice thickness 9.0 Am. Using the manufacturer's software, nascent bone formation was assessed using a global thresholding technique with threshold at 220 or threshold=0 to 2.55). Bone volume (BV), bone mineral density (BMD), trabecular (Tb) number, Tb thickness, and Tb spacing were calculated with the structural reconstruction by using the μCT software.

### Histological Analysis

Histological analysis was performed to qualitatively evaluate intramuscular bone formation after 28 days. The explanted scaffolds were fixed in 10% neutral buffered formalin overnight. The fixed samples were decalcified using a Surgipath Decalcifier II procedure, and samples were then dehydrated gradually using increasing concentrations of ethanol and then treated with xylene (Merck. Germany). Finally, samples were embedded in paraffin in a sagittal orientation and cut in 5.0 μm thick sections onto glass Superfrost Plus Slides (Fisher Scientific, Pittsburgh, Pa.) using RM2125 RT Microtome (Leica). Sections were deparaffinized and rehydrated by placing the slides in xylene, followed by graded ethanol washes and deionized water. Finally, specimens were stained with Hematoxylin-Eosin (H&E). For bright field examination of the specimens, images were acquired by Olympus Stereoscope SZX12 and an Olympus BX61 microscope, both equipped with a digital camera. All static histomorphometry analyses were performed according to standard protocols by using the OsteoMeasure XP (OsteoMetrics, Inc., Atlanta, Ga.).

### Statistical Analysis

Data are represented as mean±SEM (standard error of the mean). Statistical analysis was performed using GraphPad Prism software version 7 for windows (GraphPad Software Inc., San Diego, Calif.). Differences between experimental groups (three or more groups) were examined by using one-way analysis of variance (ANOVA) followed by Tukey's post hoc test and an unpaired two-tailed t-test was used to compare between two groups. For all experiments, P values less than or equal to 0.05 were considered significant.

### Results

### Preparation and Characterization of PEI-pDNA Nanoplexes

The PEI-(pBMP-2+pFGF-2) nanoplexes were formed by electrostatic interactions at NP ratios of 10 in order to obtain optimal transfection efficacy (Elangovan et al., 2014). PEI was used as a non-viral vector because of its high buffering capacity and its ability to offer high transfection efficacy (Boussif et al., 1995). DLS measurements showed that the nanoplexes were 117 nm (+1.5 nm) in diameter with a polydispersity index (PDI) value of less than 0.1 indicating narrow size distribution. The zeta potential of the formed nanoplexes was +30.5 mV (±0.3 mV). TEM images of the PEI-(pBMP-2+pFGF-2) nanoplexes confirmed that the nanoplexes were spherical with the average size of <67 nm (FIG. 1b). The difference between the nanoplexes size estimated by using DLS and TEM can be explained by the fact that DLS measures the hydrodynamic diameter compare to the TEM that evaluates the particles in a dehydrated state. TEM images also confirmed cellular uptake (blue arrows) and cytoplasmic distribution, when BMSCs were treated with PEI-(pBMP-2+pFGF-2) nanoplexes (FIG. 1c). TEM images also showed the osmotic swelling and rupture of endosomes (FIG. 1d), which can be explained according to the proton sponge effect. PEI protonatable amino groups exhibit considerable buffering capacity over almost the entire pH range which leads to endo-lysosomal vesicle osmotically swelling, which ultimately results in the release of the vector into the cytoplasm (Boussif et al., 1995: Akinc et al., 2005). Two desirable characteristics of the nanoplexes are their small size and positive zeta-potential which can lead to efficient cell entry by clathrin-mediated endocytosis (Wagner et al., 1991) and their endo-lysosomal escape (Godbey et al., 1999).

### Preparation and Characterization of INS MPs

INS MPs delivery system was chosen due to its ability to offer sustained release of INS as compared with INS. INS MPs were successfully prepared using a double emulsion solvent evaporation method. SEM images of INS MPs containing 10 Units of bovine INS are shown in FIG. 1e(1). SEM images demonstrated spherical particles with smooth surface covered by small pore openings on their surface which can be attributed to organic solvent diffusion from the particle core to their surface during particle solidification (Uchida et al., 1994). The loading of INS into the MPs did not affect the surface morphology when compared to the bMPs. SEM was also utilized for particle size determination which revealed that INS MPs and bMPs had a mean size of 20 μm (±4.07 μm) in diameter. The EE and DL were determined to be 66% (±4.8%) and 39.7 (+3.1) μg INS/mg MPs from three replicate studies, respectively.

### In Vitro Release Kinetics of INS MPs

The cumulative release rate of INS from INS MPs and from fibrin gel loaded with INS MPs ((INS MPs)Gel) in PBS was investigated using the Micro BCA™ Protein Assay Kit. The main goal of the release study was to ensure that the designed delivery system could provide long time interval release of the entrapped INS as well as reducing the initial burst release. The initial burst release that is usually observed in protein-loaded PLGA MPs can be a serious problem (Shively et al., 1995) with INS MPs because of narrow therapeutic window of INS and the risk of hypoglycemic shock.

The surface morphology of the INS MPs after 28 days of release was assessed using SEM. Rough surfaces with increased porosity due to the erosion of the PLGA in the aqueous environment were observed (FIG. 1e(2)). Release profiles of INS from INS MPs and ((INS MPs)Gel) are displayed in FIG. 1e(3).

During the first 24 hours an initial burst release of 38% was observed from INS MPs, followed by a more sustained release phase lasting 15 days. The release pattern displayed by INS MPs would be not favorable for a therapeutic application. In comparison, in the ((INS MPs)Gel) delivery system the initial burst release was reduced to 17% with the sustained release of INS lasting 21 days, giving us a more suitable means to locally deliver INS for extended time. The bioactivity and the efficacy of the released INS from PLGA MPs has been described (Han et al., 2012); Takenaga et al., (2004).

### Evaluation of the Biocompatibility of the Implants

The biocompatibility of the implants were examined using ZDF and ZL rats. ZDF rats were bled at 0, 7, 14, 21 and 28 days post implantation and the potential toxicity of the implants was examined using serum biomarkers. The investigated biomarkers include aspartate transaminase (AST), alanine aminotransferase (ALT), alkaline phosphatase (ALP), bilirubin, blood urea nitrogen (BUN), and creatinine. Twenty eight days post implantation, examined samples showed no evidence of toxicity (FIG. 2b) and had no significant effect on the animal weight: ZL rats gained weight albeit not significantly (FIG. 2d, 2e). In addition, the blood glucose level of the ZDF rats were monitored during the course of the study (at 7, 14, 21 and 28 days). There was no significant difference in blood glucose levels in the rats receiving either treatments (FIG. 2c). The biocompatibility of the implants was confirmed as all rats remained healthy for the entire period of the study, showing no noticeable sign of toxicity or other adverse effects. In order to avoid rejection of the implant and potential chronic inflammation in the presence of the biomimetic materials, the implant is biocompatible and formed of suitable biomaterials. The biocompatibility of the biomaterials to support the formation of the new bone tissue is directly correlated with its ability to support proliferation and differentiation of the host cell, and provides a platform for extracellular matrix formation without any toxic or injurious effect.

### Evaluation of In Vivo Osteoblastic Gene Expression

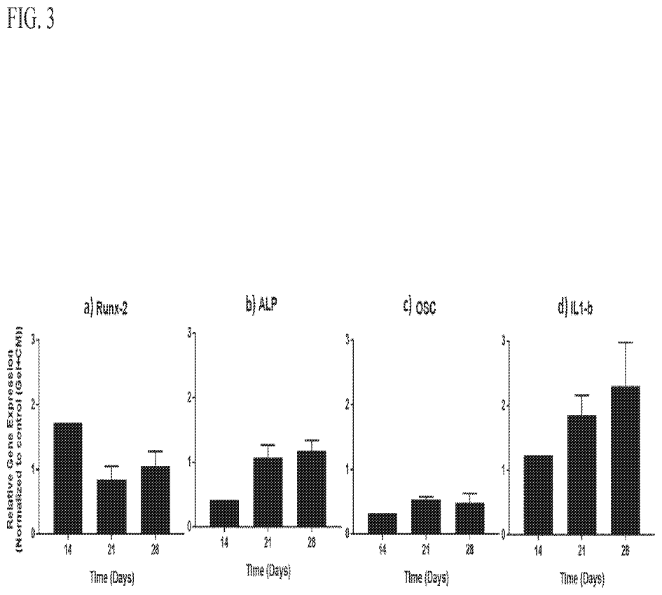

The expression of genes involved in osteogenesis in the combinatorial ((INS MPs+VD3)Gel+GAM) group was quantitatively evaluated by performing RT-qPCR on total RNA extracted from explanted ((INS MPs+VD3)Gel+GAM) scaffolds. The expression of target genes at 14, 21, and 28 days post implantation was compared to mRNA levels at 14, 21 and 28 days post implantation of control group receiving (Gel+CM) treatment. The relative gene expression values are shown in FIG. 3.

Runt-related transcription factor-2 (Runx-2) shows almost one order of magnitude increase, 14 days post implantation, then RUNX-2 expression was down regulated until day 28 (FIG. 3a). Alkaline phosphatase (ALP) demonstrated reduced expression during week 2 (with negative regulation peak), however ALP expression slightly increased over time showing a distinctive peak of expression at day 28 with approximately 0.2 orders of magnitude increase in mRNA (FIG. 3b). Osteocalcin (OSC) demonstrated reduced expression with peaks of negative regulation during 28 days (FIG. 3c). Interleukin-1 beta (IL1-b) expression increased during the entire period with a positive modulation of up to 2 orders of magnitude increase in mRNA expression during the 28 days (FIG. 3d).

IL1-b has been shown to play a role in recruiting inflammatory cells, stimulating angiogenesis, enhancing extracellular matrix synthesis, and promoting the formation of the cartilaginous callus (Kon et al., 2001). IL1-b has been shown previously to have a biphasic expression pattern during bone healing, where within the first 24 hours, macrophages express high concentration of IL1-b, then its concentration declines to undetectable levels by day 3 (Mountziaris et al., 2008). The expression of IL1-b rises again approximately 3 weeks following the injury, mainly due to expression by osteoblasts. High concentrations of IL1-b stimulate proteases to degrade callus tissue and help with bone remodeling (Kon et al., 2001); Mountziaris et al., 2008). The upregulation of the IL1-b expression within 28 days suggest the presence of the osteoblasts and perhaps bone remodeling. Further studies may evaluate the expression levels of Runx-2, ALP, and OSC at earlier time points including 3, 5, 7 and 10 days post implantation, since these gene expressions may be detected from day 5 and then increased by day 14 (Gerstenfeld et al., 2003).

### Assessment of In Vivo Bone Regeneration

The translational potential of the composite material was investigated in vivo in a challenging diabetic rat model. The synergistic effect on ectopic bone formation following delivery of PEI-(pBMP-2+pFGF-2) nanoplexes, INS and VD3 after 28 days of implantation was studied in vivo quantitatively and qualitatively. High resolution micro-computed tomography (μCT) scanning was carried out to quantitatively and qualitatively assess the newly-formed bone tissue within the intramuscular (IM) pockets at 28 days post implantation.

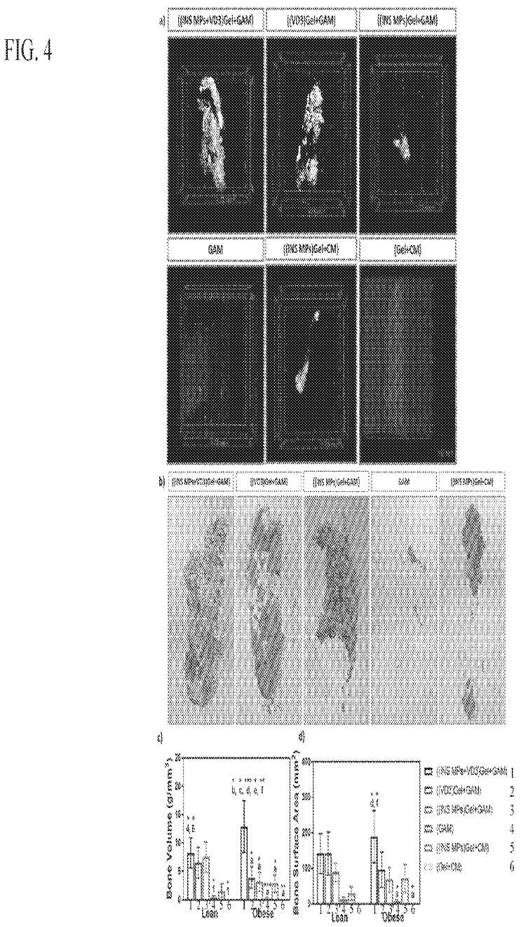

The μCT qualitative assessment of explanted constructs demonstrated an increase in mineralized tissue portion and callus formation in the group that was treated with ((INS MPs+VD3)Gel+GAM), ((VD3)Gel+GAM) or ((INS MPs)Gel+GAM) compared to the groups treated with GAM, ((INS MPs)Gel+CM) or (Gel+CM). The μCT images rarely showed newly regenerated bone in the groups treated with GAM or (Gel+CM) (FIG. 4a, 4b). This was confirmed by a quantitative analysis where the amount of de novo bone formation was assessed by analyzing the mineralized bone volume and bone surface area. In obese (ZDF) rats, there was significantly increased bone volume in the combinatorial ((INS MPs+VD3)Gel+GAM) group compared to all other treatment groups (FIG. 4c). Furthermore, the bone surface area showed the same trend that was observed with bone volume assessment. As shown in FIG. 4d in obese rats, the regenerated bone surface area was significantly higher in the group that was treated with ((INS MPs+VD3)Gel+GAM) compared to the GAM or (Gel+CM) group. In the lean (ZL) rats, relative to GAM and (Gel+CM) the presence of the INS MPs and VD3 together or individually in combination with GAM enhanced bone formation IM. There was significantly increased bone volume in the combinatorial ((INS MPs+VD3)Gel+GAM) group compared to GAM and (Gel+CM) treatment groups (FIG. 4c). A trend toward higher bone surface area of the regenerated bone was observed in the groups that received ((INS MPs+VD3)Gel+GAM), ((VD3)Gel+GAM) and ((INS MPs)Gel+GAM) treatments compared to the GAM or (Gel+CM) group, albeit not significantly (FIG. 4d). Also, the presence of plasmid loaded nanoplexes provided an enhancement in bone regeneration (0.5 fold increase) when compared to the collagen matrix alone (Gel+CM).

### Example 2

Various variants of the protein and nucleic acid sequences described in Example 1 and Table 2 herein can be used in various embodiments. For example, variants of the growth factors encoded by the genes listed in Table 2 can be used in various embodiments. Variants of the growth factors can include fragments of the growth factors and/or variants that have substantial identity to the growth factors. Table 2 includes the gene symbol and gene ID, obtained from the HGNC database on Jul. 17, 2018 (https://www.genenames.org/. HGNC—HUGO Gene Nomenclature Committee, HUGO—Human Genome Organisation) for each growth factor.

TABLE 2

| BMP1 1067 | BMP15 1068 | FGF10 3666 | FGF21 3678 | PDGFD |
|---|---|---|---|---|
| | | | | 30620 |
| BMP2 1069 | FGF1 3665 | FGF11 3667 | FGF22 3679 | TGFB1 |
| | | | | 11766 |
| BMP3 1070 | FGF2 3676 | FGF12 3668 | FGF23 3680 | TGFB2 |
| | | | | 11768 |
| BMP4 1071 | FGF3 3681 | FGF13 3670 | IGF1 5464 | TGFB3 |
| | | | | 11769 |
| BMP5 1072 | FGF4 3682 | FGF14 3671 | IGF2 5466 | VEGFA |
| | | | | 12680 |
| BMP6 1073 | FGF5 3683 | FGF16 3672 | HGF 4893 | VEGFB |
| | | | | 12681 |
| BMP7 1074 | FGF6 3684 | FGF17 3673 | NGF 7808 | VEGFC |
| | | | | 12682 |
| BMP8A | FGF7 3685 | FGF18 3674 | PDGFA 8799 | VEGFD |
| 21650 | | | | 3708 |
| BMP8B 1075 | FGF8 3686 | FGF19 3675 | PDGFB 8800 | |
| BMP10 | FGF9 3687 | FGF20 3677 | PDGFC 8801 | |
| 20869 | | | | |

### Example 3

Various forms of bioavailable vitamin D can be used in various embodiments. Bioavailable vitamin D includes various vitamin D analogs. Bioavailable vitamin D includes but is not limited to calcitriol, ercalcitriol, calcipotriol, aricalcitol, doxercalciferol, falecalcitriol, maxacalcitol, tacalcitol, alfacalcidol, eldecalcitol, seocalcitol, 20-epi-1,25(OH)₂D₃, lexicalcitol, 20-epi-1,25(OH)₂D₃, CD578, inecalcitol, TX527, or ILX23-7553.

### Example 4

### 1. Materials and Methods

### 1.1. Materials:

Resomer® RG503 (PLGA 50:50, IV 0.32-0.44 dL/g) was obtained from Boehringer Ingelheim Pharma Gmbh & Co (Ridgefield, Conn.). Poly(vinyl alcohol) (PVA; Mowiol® 8-88) was purchased from Sigma-Aldrich® (St. Louis, Mo.). Insulin from bovine pancreas powder, and Cholecalciferol (activated VD3) were acquired from Sigma-Aldrich. Branched PEI (mol. wt. 25 kDa) and the GenElute™ HP endotoxin-free plasmid maxiprep kit were purchased from Sigma-Aldrich. Plasmid DNA (6.9 Kb) encoding BMP-2 protein and plasmid DNA (4.9 Kb) encoding FGF-2 were purchased from Origene Technologies, Inc. (Rockville, Md.). Absorbable type-I bovine collagen was purchased from Zimmer Dental Inc. (Carlsbad. Calif.). TISSEEL™ Fibrin Sealant was obtained from Baxter Healthcare Corp (Deerfield, Ill.). The RNeasy Mini Kit was purchased from Qiagen Inc (Germantown, Md.). The TaqMan Reverse Transcription Reagents and 18S-rRNA were purchased from Applied Biosystems (Foster City, Calif.). All primers were obtained from Integrated DNA Technologies (Coralville, Iowa). Micro BCA™ Protein Assay Kit and RNAlater™ Stabilization Solution was obtained from Thermo Scientific (Pittsburgh, Pa.). Human bone marrow stromal cells (BMSCs) were purchased from the American Type Culture Collection (ATCC®, Manassas, Va.). Dulbecco's Modified Eagle's Medium (DMEM), try psin-EDTA (0.25%, 1× solution) and Dulbecco's phosphate buffered saline (PBS) were purchased from Gibco® (Invitrogen™, Grand Island, N.Y.). Fetal bovine serum (FBS) was obtained from Atlanta Biologicals® (Lawrenceville, Ga.). Gentamycin sulfate (50 mg/ml) was purchased from Mediatech Inc. (Manassas, Va.). All other chemicals and solvents used were of reagent grade from Sigma Aldrich.

### 1.2. Bone Marrow Stromal Cells (BMSCs) Culture:

BMSCs were cultured and maintained in DMEM (supplemented with 10% FBS, 1 mM Glutamax™ (Gibco), 1 mM sodium pyruvate (Gibco), and 1% gentamycin (50 μg/ml)) in a humidified incubator at 37° C. and 5% CO₂ flow (Sanyo Scientific Auto flow. Infrared direct heat CO₂ incubator). BMSCs were passaged using 0.25% trypsin-EDTA (Invitrogen™). In this study, BMSCs were used at passages 3 to 4. Cells were cultured on 75 cm² polystyrene cell culture flasks (Corning, N.Y., USA). The BMSCs were mycoplasma-free as determined by a MycoAlert mycoplasma detection kit (Lonza, Morristown, N.J.).

### 1.3. Composite Design and Fabrication

Isolation of Plasmid DNA (pDNA) Encoding BMP-2, and FGF-2 and Fabrication of PEI-pDNA Nanoplexes:

To amplify the plasmid, the pDNAs encoding BMP-2, and FGF-2 were independently transformed into chemically competent E. coli DH5α™. Subsequently pDNAs were extracted, purified and analyzed for purity. Then, PEI-pDNA nanoplexes (200 μL) containing of 25 μg of pFGF-2 and 25 μg of pBMP-2 were fabricated at a molar ratio of PEI amine (N) to pDNA phosphate (P) groups of 10.

Preparation of Gene Activated Matrixes (GAMs):

Absorbable type-I bovine collagen was cut into cylindrical scaffolds (radius=5 mm; height=5 mm), then a 4 mm sterile biopsy punch was used to remove the central core of the scaffold, yielding a ring-shaped construct. Afterwards, the PEI-(pBMP-2+pFGF-2) nanoplex solution (200 L) was injected into the collagen scaffolds using a sterile 28 gage needle and then the GAMs were frozen at −20° C. until required. Collagen matrices (CM) injected with 200 μL of sterile water (RNase and DNase free) were used as controls.

Fabrication of INS Containing PLGA MPs (INS MPs):

INS MPs, e.g., from 25 nm to 100 nm, 100 nm to 1 micron, 1 to 100 microns in diameter, were prepared using the water-in-oil-in-water (W/O/W) double emulsion method. Briefly, 12.5 mg of lyophilized INS powder was dissolved in 200 μL 0.01 N HCl, and the pH adjusted to 4.0. Aqueous INS solution was mixed with 1.5 mL of dichloromethane (DCM) containing 200 mg of PLGA, then sonicated at an energy output level of 40% amplitude for 30 s. The primary emulsion was then re-emulsified with 30 mL of 1% PVA aqueous solution using a homogenizer at 6500 rpm for 30 s. The W/O/W emulsion was stirred for 1.5 h at room temperature, allowing the DCM to evaporate. The emulsion gradually solidified as the solvent diffused from the emulsion droplets into the external phase. The resulting MPs were collected by centrifugation using at 29×g for 5 min, resuspended in 30 mL of Nanopure sterile water, and washed twice with Nanopure sterile water (Thermo Scientific™ Nanopure™). Particles were then suspended in 5 mL of Nanopure sterile water which was frozen at −20° C. for 4 h and lyophilized for 18 h at collector temperature of −53° C. and 0.08 mBar pressure using a FreeZone 4.5-L Benchtop Freeze Dry System (Labconco Corporation, Kansas City, Mo.). In this study. INS treatments were provided as INS encapsulated in MPs which will be referred as INS MPs for simplification and the control blank PLGA particles are referred to as bMPs.

Preparation of Fibrin Gel Loaded with INS MPs and VD3:

The fibrin sealant TISSEEL kit was used, which is composed of sealer protein solution (100 mg/mL fibrinogen) and thrombin solution (500 units/mL thrombin). Fibrin gels loaded with INS MPs and VD3 were prepared by diluting fibrinogen solution in HBSS buffer to obtain 12.5 mg/mL fibrinogen solution. Then I1 mg of INS MPs (10 units, equivalent of 0.455 mg of INS) and 5 μg of the active form of VD3 were added to the thrombin solution to form a suspension containing 10 units/mL of thrombin. Finally, the two components were mixed simultaneously at a 1:1 ratio, forming a fibrin clot on delivery. In both control and/or treatment groups fibrin gel was prepared as described and was incorporated into the GAM core.

Fabrication of Final Composite (GAMs Loaded with INS MPs and VD3 Gel):

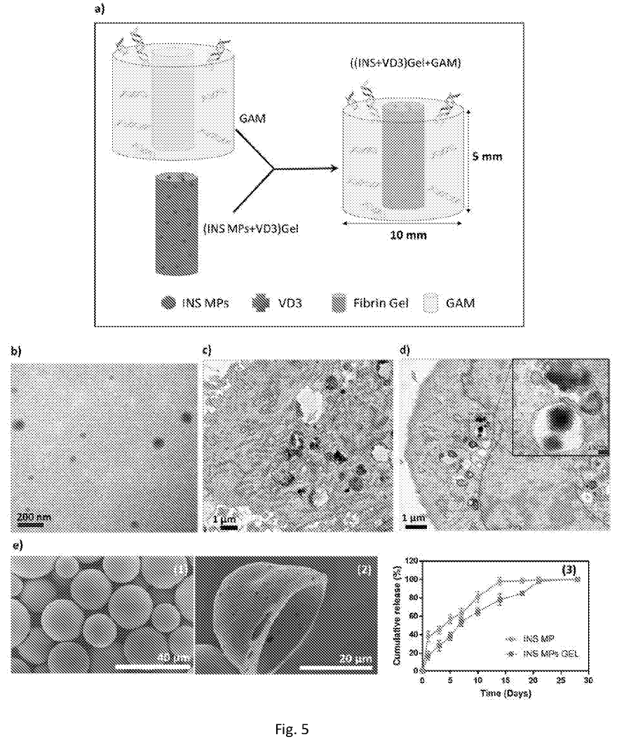

The final construct was prepared by injection of fibrin clot into the ring-shaped GAMs (collagen scaffold containing nanoplexes of pDNA (pBMP-2+pFGF-2)). Then the implants were frozen at −20° C. until required (FIG. 5a).

### 1.4. Size and Zeta Potential Measurements

Using a Zetasizer Nano ZS particle analyzer, via dynamic light scattering (DLS) technique, particle size and zeta potential of nanoplexes were measured (Malvern Instrument Ltd., Southborough, Mass.). Using an aqueous solution of the nanoplexes the size was measured at 1730 backscatter detection in disposable polystyrene cuvettes and zeta potential was measured in a zeta potential folded capillary cell at 25° C.

### 1.5. Microscopic Evaluation of the MPs and Nanoplexes

SEM. The surface morphology of the INS MPs was performed after fabrication and also at the end of the release study using a scanning electron microscope (SEM, Hitachi S-4000, Schaumburg, Ill.). Briefly, 0.05 mg/mL INS MPs were added onto silicon wafers and air-dried for 24 h and the wafers were then placed on adhesive carbon tabs mounted on SEM specimen stubs. All the specimen stubs were sputter-coated with approximately 5 nm of gold/palladium by ion beam evaporation (argon-beam K550 sputter coater (Emitech Ltd). Images were captured using the SEM operated at 5 kV accelerating voltage (S-4800, Hitachi High-Technologies).

TEM. The shape of PEI-(pBMP-2+pFGF-2) nanoplexes prepared at N/P ratio of 10 as well as nanoplex uptake by BMSCs were visualized by transmission electron microscopy (TEM, JEOL JEM-1230) equipped with a Gatan UltraScan 1000 2 k×2 k CCD acquisition system (JEOL USA Inc.). In short, 10 μL of the PEI-pDNA nanoplexes (containing of 25 μg of pFGF-2 and 25 μg of pBMP-2) was absorbed onto carbon-coated grids for 30 s (400-mesh TEM carbon grid by Auto 306 BOC Edwards). All of the TEM grids were pre-coated with a Formvar solution (0.5%) in an ethylene dichloride film ((Electron Microscopy Sciences (EMS)). Then, using Whatman filter paper, the excess sample liquid was removed and the grids were air dried.

Using TEM the cellular uptake of nanoplexes was examined. BMSCs were seeded at 10⁵ cells/well into 12 well plates for 24 h. BMSCs then were incubated with 20 μl (1 μg pDNA) of complexes (N/P ratio of 10) for 4 h in the presence of serum-free medium. Then the serum-free medium was replaced with growth medium containing serum. At 48 h post transfection, BMSCs were fixed for 30 min with glutaraldehyde (2.5%) in a sodium cacodylate buffer (0.1 M, pH 7.4, (EMS)). Afterwards, cells were rinsed twice for 4 min each with cacodylate buffer (0.1 M, pH 7.4, (EMS)). Then to improve the efficacy of the fixation and escalate the electron density, BMSCs were treated with 1% osmium tetroxide (EMS) for 30 min. The fixed BMSCs were then stained with uranyl acetate (2.5%. (EMS)) for 5 min, following a double wash with distilled water. Samples were then dehydrated gradually by sequentially incubating in 25%, 50%, 75% and 95% ethanol for 4 min per solution, followed by two final 5 min incubations with 100% ethanol. Finally, to embed the dehydrated samples in Epon (Ted Pella Inc.), samples were infiltrated with a mixture (1:1) of ethanol: Epon for 30 min, and then fixed in Epon for 8 h at 70° C. Using a Leica EM UC6 Ultramicrotome MZ6 (Reichert Technologies, Buffalo, Ny), thin sections of samples were prepared (50-70 nm). Finally, these sections were mounted on a Formvar-coated 400-mesh TEM carbon grid, and the images were obtained using TEM, JEOL JEM-1230.

### 1.6. Quantification of INS Loading and Encapsulation Efficiency of INS MPs

To quantify INS loading, INS MPs (7.1 mg) was dissolved in chloroform (1 mL), and then mixed with 0.01 N HCL (2 mL) and shaken vigorously to allow the active ingredient migrate to the aqueous phase for at least 30 min. The aqueous phase (top layer) was collected and neutralized with 1N NaOH (pH 7.0). Then drug loading (DL) and encapsulation efficiency (EE) were determined using the Micro BCA™ Protein Assay Kit following the manufacturer's recommended protocol (Thermo Scientific). DL and EE were calculated according to the following equations.

$$Drug Loading ( \frac{drug ( µg )}{MPs ( mg )} ) = \frac{Weight of insulin entrapped within MPs ( µg )}{Total weight of MPs ( mg )}$$

$$Encapsulation Efficiency ( \% ) = \frac{Weight of insulin entrapped within MPs ( mg )}{Total weight of initial insulin ( mg )} \times 100$$

### 1.7. In Vitro Kinetics of Insulin Release

Insulin release directly from INS MPs and from fibrin gel containing INS MPs was investigated using Micro BCA™ Protein Assay. For assessment of the INS release from INS MPs and gel approximately 10.5 mg of INS MPs was added in 1 mL of PBS (IX) into a 2 mL micro-centrifuge tube or by completely submerging the fibrin gel (100 μL) containing INS MPs (10.5 mg) in I mL of PBS (1×) into scintillation vial. The samples were incubated under shaking (300 rpm) at 37° C. For the direct release of INS from INS MPs at regular time intervals, micro-centrifuge tubes were centrifuged at 180×g for 8 min, supernatant was harvested for further analysis and INS MPs were resuspended in fresh PBS. To quantify the INS release from fibrin gels containing INS MPs, at regular time intervals supernatants were taken and replaced with fresh PBS. And the amount of released INS was estimated using the Micro BCA™ Protein Assay Kit according to the manufacturing protocol. All samples were analyzed in triplicate and stored at −20° C. until further analysis.

### 1.8 Animal Models and Surgical Plan

The study was approved by, and conducted according to guidelines established by, the University of Iowa Institutional Animal Care and Use Committee (IACUC), Iowa. Adult male 10-12 week old Zucker diabetic fatty (ZDF) rates weighing −0.34 kg and Zucker lean (ZL) rats weighing −0.3 kg were purchased from Charles River Laboratories (Wilmington, Mass.) and housed and cared for in the animal facilities. The rats were maintained on a heating pad (37° C.) and were anesthetized by continuous isoflurane inhalation through a vaporizer (0.5-5.0%) prior to implantation and the surgical sites were shaved and disinfected with a 30% betadine solution.

Lumbar Paraspinal Sites:

A mid-line skin incision was made of approximately 1 cm from the cranial crest to the iliac crest and bupivacaine (0.5%) was dripped onto the muscle prior to dissection. The paraspinous muscle was exposed and an incision, approximately 1.5 cm in length, was made through the fascia and the underlying muscles and separated to create a pocket for implantation. The procedure was repeated on the contralateral side of the spine. Following implant placement, soft tissues and skin were closed in layers using absorbable suture material.

Bicep Femoris Sites:

An approximately 1.5 cm skin incision was made over the biceps femoris muscle on both limbs and bupivacaine (0.5%) was dripped on the muscle prior to dissection. Pockets were made between the biceps femoris and vastus lateralis by longitudinal blunt and sharp dissection, without cutting the muscles. Following implant placement, the skin incisions were closed using absorbable suture material.

### 1.9. Experimental Design

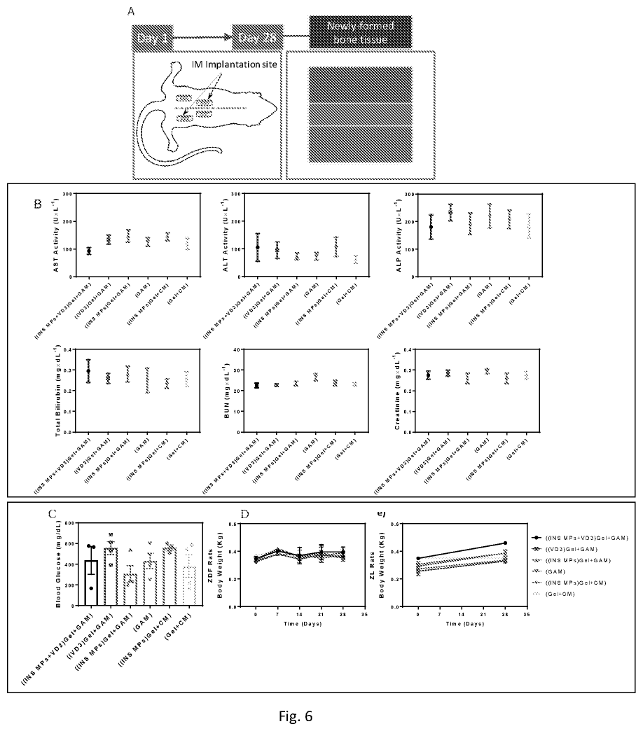

Scaffolds were implanted (four implants per rat) into intramuscular (IM) pockets in the lumbar paraspinal and bicep femoris sites of the animals. One in each biceps femoris muscle (right and left leg), and one in each dorsal paraspinous muscle (right and left side) (FIG. 6a). Animals were randomly assigned to the six following treatment groups: 1) Implant 1: ((INS MPs+VD3)Gel+GAM), (n=40); 2) Implant 2: ((VD3)Gel+GAM), (n=40); 3) Implant 3: ((INS MPs)Gel+GAM), (n=40); 4) Implant 4: GAM, (n=40): 5) Implant 5: ((INS MPs)Gel+CM), (n=40): and 6) Implant 6: (Gel+CM), (n=40). Animals were monitored twice daily during postoperative recovery for any clinical signs of illness, fracture, or reaction to treatment. At 14, 21, and 28 days after surgery, animals were euthanized by intracardiac injection of Euthasol, and the implantation sites with surrounding bone were removed and collected for subsequent analysis.

### 1.10. Weight and Blood Sample Analysis

Weights of individual rats were monitored and recorded at 7, 14, 21, and 28 days post-surgery. In addition, blood samples were collected from the tail prior to treatment and at the end of the treatment regimen (7, 14, 21, and 28 days after surgery). The blood serum was separated by centrifugation (1000×g at 4° C. for 10 min) and stored at −80° C. Serum samples were shipped to IDEXX Laboratories (Sacramento, Calif.) for toxicity analyses.

### 1.11. RNA Extraction and Quantitative Real Time Polymerase Chain Reaction (Q-PCR)

On days 14, 21, and 28 scaffolds were explanted and stored in RNAlater™ Stabilization Solution. Scaffolds were submerged in 5 volumes of RNAlater solution (1 g tissue required 5 mL of solution), and stored at 4° C. overnight. The supernatant was then removed and samples were stored at −80° C. until ready for use. Frozen implants were placed in an RNase-free mortar and pestle and ground into a powder while immersed in liquid nitrogen. Then the total RNA was extracted using the RNeasy Mini Kit (Qiagen) according to the manufacturer's instructions. The integrity of the purified RNA (RNA integrity, RIN) was then assessed by using the Agilent 2100 Bioanalyzer system (Agilent Technologies, Santa Clara, Calif.). Purified RNA was then reversely transcribed with random hexamers using High-Capacity cDNA Reverse Transcription kit (Applied Biosystems) in the thermocycle system (Bio-Rad, Hercules, Calif.). The expression levels of genes involved in osteogenesis were then investigated using the TaqMan Universal PCR Master Mix on QuantStudio 3 Real-Time PCR System. Quantitative PCR was carried out using the primers and probes listed in Table 3 with hypoxanthine guanine phosphoribosyl transferase (HRPT) and ubiquitin C (UBC) as the internal controls. Each 20 μL PCR reaction well contained 2 μL of cDNA, 1 μL of primer-probe mix, and 10 μL of 2× PrimeTime® Gene Expression master mix with a ROX passive reference dye. Cycling conditions were 50° C. for 2 min, and 90° C. for 3 min followed by 40 cycles of 90° C. for 15 s (denaturation) and 60° C. for 1 min (annealing and extension). Analysis of data was performed using the auto-threshold baseline and the 2^{-ΔΔCt} method. The expression levels of the target genes were normalized to the expression levels of the house keeping genes. Each sample was run in duplicate and values represent the mean of at least 2 replicates.

### 1.12. Transcription Profiling Using RNA-Seq

Transcription profiling using RNA-Seq was performed by the University of Iowa Genomics Division using manufacturer recommended protocols. Initially, 500 ng of DNase I-treated total RNA was used to enrich for polyA containing transcripts using oligo(dT) primers bound to beads. The enriched mRNA pool was then fragmented, converted to cDNA and ligated to sequencing adaptors containing indexes using the Illumina TruSeq stranded mRNA sample preparation kit (Cat. #RS-122-2101, Illumina, Inc., San Diego, Calif.). The molar concentrations of the indexed libraries were measured using the 2100 Agilent Bioanalyzer and combined equally into pools for sequencing. The concentration of the pools were measured using the Illumina Library Quantification Kit (KAPA Biosystems, Wilmington, Mass.) and sequenced on the Illumina HiSeq 4000 genome sequencer using 150 bp paired-end SBS chemistry.

### 1.13. RNA-Seq Mapping and Analysis

Raw sequencing reads were aligned to the R. norvegicus genome m6 using STAR version 2.2.1 (Dobin 2013). On average, samples had 55M reads (range 38-72M), with 73.0% of reads mapped uniquely, 10.3% mapped to multiple loci, and 15.9% too short to map. Reads were quantified at the gene level against known transcripts from Ensembl release 89, requiring correct strand orientation. Of 32883 genes assessed, 14423 were above the minimum expression threshold of 1 count per million (cpm) reads in at least one sample. Expression differences were qualitatively assessed as base-2 log ratios (with a +0.1 cpm prior to expression) against the control condition, Gel-CM.

TABLE 3
Probe and primer sequences.

| | Probe | Forward | Reverse |
|---|---|---|---|
| Runx-2 | TGA AAC TCT | GCC AGG TTC | CGT CCA CTG |
| | TGC CTC GTC | AAC GAT CTG | TCA CTT TAA |
| | CGC TC (SEQ | A (SEQ ID | TAG CTC |
| | ID NO: 20) | NO: 21) | (SEQ ID NO: |
| | | | 22) |
| OSC | CCA GCA GAG | AGA CCT AGC | GCT TGG ACA |
| | TGA GCA GAG | AGA CAC CAT | TGA AGG CTT |
| | AGA GG (SEQ | GA (SEQ ID | TG (SEQ ID |
| | ID NO: 23) | NO: 24) | NO: 25) |
| ALP | TCT GGA ACC | AAA CCT AGA | ICC GAT TCA |
| | GCA CTG AAC | CAC AAG CAC | ACT CAT ACT |
| | TGC T (SEQ | TCC (SEQ ID | GCA T (SEQ |
| | ID NO: 26) | NO: 27) | ID NO: 28) |
| IL1-b | TGG CTT ATG | GIG CTG TCT | TTG TCG TTG |
| | TTC TGT CCA | GAC CCA TGT | CTT GTC TCT |
| | TTG AGG TGG | (SEQ ID NO: | CC (SEQ ID |
| | (SEQ ID NO: | 30) | NO: 31) |
| | 29) | | |
| HRPT | TGG ATA CAG | GGT GAA AAG | GCT TTT CCA |
| | GCC AGA CTT | GAC CTC TCG | CTT TCG CTG |
| | TGT TGG ATT | AAG (SEQ ID | ATG (SEQ ID |
| | (SEQ ID NO: | NO: 33) | NO: 34) |
| | 32) | | |
| UBC | CCC AAG AAC | GAC AGG CAA | AAA ACT AAG |
| | AAG CAC AAG | GAC CAT CAC | ACA CCT CCC |
| | AAG GGC | TC (SEQ ID | CAT C (SEQ |
| | (SEQ ID NO: | NO: 36) | ID NO: 37) |
| | 35) | | |

### 1.14. Histological Observation and Analysis

Histological analysis was performed to qualitatively evaluate intramuscular bone formation after 28 days. The explanted scaffolds were fixed in 10% neutral buffered formalin overnight. The fixed samples were decalcified using a Surgipath Decalcifier II procedure, and samples were then dehydrated gradually using increasing concentrations of ethanol and then treated with xylene (Merck. Germany). Finally, samples were embedded in paraffin (EM-400, Surgipath (Leica Biosystems Inc. Lincolnshire, Ill.). Samples were sectioned at 5 μm in thickness onto glass Superfrost Plus Slides (Fisher Scientific, Pittsburgh, Pa.) using a RM2125 RT Microtome (Leica). Sections were deparafinized and rehydrated by placing the slides in xylene, followed by graded ethanol washes and deionized water. Finally, specimens were stained with Hematoxylin-Eosin (H&E). For bright field examination of the specimens, images were acquired by Olympus Stereoscope SZX12 and an Olympus BX61 microscope, both equipped with a digital camera. All static histomorphometry analyses were performed according to standard protocols by using the OsteoMeasure XP (OsteoMetrics, Inc., Atlanta, Ga.).

### 1.15. Micro-Computed Tomography

The three-dimensional x-ray micro-computed tomography (μCT) imaging was performed to quantitate the ectopic bone formation in the presence of various treatments. High resolution Skyscan 1176 (Kontich, Belgium) was used with the following settings: voltage 50 KeV, current 500 μA, exposure 1050 ms and slice thickness 9.0 μm. Using the manufacturer's software, nascent bone formation was assessed using a global thresholding technique with threshold=90 to 255). Bone volume (BV), and bone surface area were calculated with the structural reconstruction by using the μCT software.

### 1.16. Statistical Analysis

Data are represented as mean±SEM (standard error of the mean). Statistical analysis was performed using GraphPad Prism software version 7 for windows (GraphPad Software Inc., San Diego, Calif.). Differences between experimental groups (three or more groups) were examined by using one-way analysis of variance (ANOVA) followed by Tukey's post hoc test and an unpaired two-tailed t-test was used to compare between two groups. For all experiments, P values less than or equal to 0.05 were considered significant.

### 3. Results and Discussion

### 3.1. Preparation and Characterization of PEI-pDNA Nanoplexes

The PEI-(pBMP-2+pFGF-2) nanoplexes were formed by electrostatic interactions at N/P ratios of 10 in order to obtain optimal transfection efficacy. PEI was used as a non-viral vector because of its high buffering capacity and its ability to offer high transfection efficacy. DLS measurements showed that the nanoplexes were 117 nm (±1.5 nm) in diameter with a polydispersity index (PDI) value of less than 0.1 indicating narrow size distribution. The zeta potential of the formed nanoplexes was +30.5 mV (±0.3 mV). TEM images of the PEI-(pBMP-2+pFGF-2) nanoplexes confirmed that the nanoplexes were spherical with the average size of <67 nm (FIG. 5b). The difference between the nanoplexes size estimated by using DLS and TEM can be explained by the fact that DLS measures the hydrodynamic diameter compared to the TEM that evaluates the particles in a dehydrated state. TEM images also confirmed cellular uptake (blue arrows) and cytoplasmic distribution, when BMSCs were treated with PEI-(pBMP-2+pFGF-2) nanoplexes (FIG. 5c). TEM images also showed the osmotic swelling and rupture of endosomes (FIG. 5d), which can be explained according to the proton sponge effect. PEI protonatable amino groups exhibit considerable buffering capacity over almost the entire pH range which leads to endo-lysosomal vesicles osmotically swelling, which ultimately results in the release of the vector into the cytoplasm. Two characteristics of the nanoplexes are their small size and positive zeta-potential which are desirable for efficient cell entry by clathrin-mediated endocytosis and their endo-lysosomal escape.

### 3.2. Preparation and Characterization of INS MPs

INS MPs was chosen as a delivery system due to its ability to offer sustained release of INS as compared with soluble INS. INS MPs were successfully prepared using a double emulsion solvent evaporation method. An SEM image of freshly prepared INS MPs containing 10 Units of bovine INS is shown in FIG. 5e(1) and demonstrates the particles to be spherical and having smooth surfaces with small pore openings/indentations likely due to organic solvent diffusion from the particles during particle solidification. The loading of INS into the MPs did not affect the surface morphology when compared to the bMPs (not shown). SEM was also utilized for particle size determination which revealed that INS MPs and bMPs had a mean size of 20 μm (±4.07 μm) in diameter. The EE and DL were determined to be 66% (±4.8%) and 39.7 (±3.1) μg INS/mg MPs from three replicate studies, respectively.

### 3.3. In Vitro Release Kinetics of INS MPs

The cumulative release of INS from INS MPs and from fibrin gel loaded with INS MPs ((INS MPs)Gel) in PBS was investigated using the Micro BCA™ Protein Assay Kit. The main goal of the release study was to ensure that the designed delivery system could provide long time interval release of the entrapped INS as well as reducing the initial burst release. The initial burst release that is usually observed in protein-loaded PLGA MPs can be a problem with INS MPs because of the narrow therapeutic window of INS and the risk of hypoglycemic shock.

In one embodiment, the nucleic acid is released in I to 12 hours, 12 to 24 hour or for up to 7 days. In one embodiment, the INS is released in 1 to 12 hours, 1 to 7 days or up to weeks, e.g., up to 3 weeks. In one embodiment, the VD3 is released in 1 to 12 hours, or 1 to 5 days.

The surface morphology of the INS MPs after 28 days of release was assessed using SEM. Rough surfaces with increased porosity due to the erosion of the PLGA in the aqueous environment were observed (FIG. 5e(2)). Release profiles of INS from INS MPs and ((INS MPs)Gel) are displayed in FIG. 5e(3). During the first 24 hours an initial burst release of 38% was observed from INS MPs, followed by a more sustained release phase lasting 15 days. The release pattern displayed by INS MPs would be not favorable for a therapeutic application. In comparison, in the ((INS MPs)Gel) delivery system the initial burst release was reduced to 17% with the sustained release of INS lasting 21 days, giving us a more suitable means to locally deliver INS for extended time. The bioactivity and the efficacy of the released INS from PLGA MPs has been previously characterized.

### 3.4. Evaluation of the Biocompatibility of the Implants

The biocompatibility of the implants was examined using ZDF and ZL rats. ZDF rats were bled at 0, 7, 14, 21 and 28 days post implantation and the potential toxicity of the implants was examined using serum biomarkers. The investigated biomarkers include aspartate transaminase (AST), alanine aminotransferase (ALT), alkaline phosphatase (ALP), bilirubin, blood urea nitrogen (BUN), and creatinine. Twenty-eight days post implantation, examined samples showed no evidence of toxicity (FIG. 6b) and had no significant effect on the animal weight; ZL rats gained weight albeit not significantly (FIG. 6d, 6e). In addition, the blood glucose levels of the ZDF rats were monitored during the course of the study (at 7, 14, 21 and 28 days). There was no significant difference in blood glucose levels in the rats receiving either treatment (FIG. 6c). The biocompatibility of the implants was confirmed as all rats remained healthy for the entire period of the study, showing no noticeable signs of toxicity or other adverse effects. In order to avoid rejection of the implant and potential chronic inflammation in the presence of the biomimetic materials, the implant is biocompatible and is formed of suitable biomaterials. The biocompatibility of the biomaterials to support the formation of the new bone tissue is directly correlated with its ability to support proliferation and differentiation of the host cells, and provides a platform for extracellular matrix formation without any toxic or injurious effect.

### 3.4. Evaluation of In Vivo Osteoblastic Gene Expression

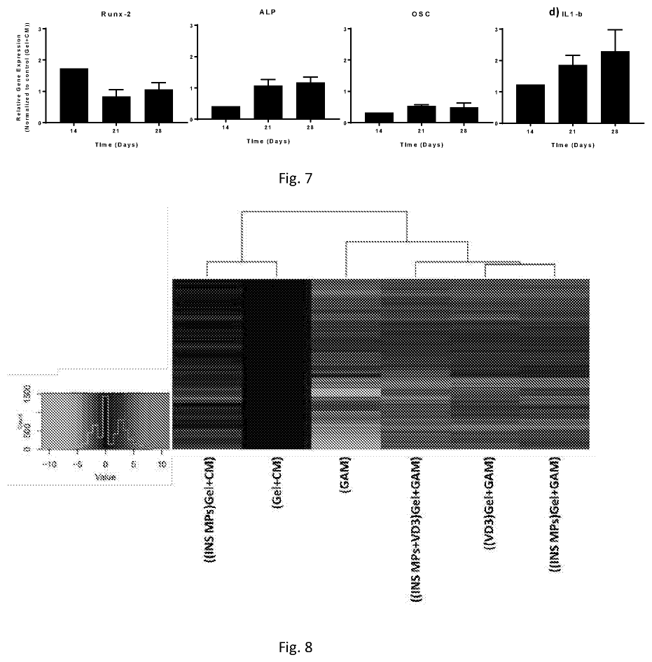

The expression of genes involved in osteogenesis in the combinatorial ((INS MPs+VD3)Gel+GAM) group was quantitatively evaluated by performing RT-qPCR on total RNA extracted from explanted ((INS MPs+VD3)Gel+GAM) scaffolds. The mRNA expression levels of target genes at 14, 21, and 28 days post implantation was compared to the control group treated with (Gel+CM). The relative gene expression values are shown in FIG. 7.

Runt-related transcription factor-2 (Runx-2) showed an almost 2-fold increase 14 days post implantation; then Runx-2 expression returned to levels comparable to the control group by days 21 and 28 (FIG. 7a). Alkaline phosphatase (ALP) demonstrated reduced expression on day 14, and then returned to levels comparable to the control group by days 21 and 28 (FIG. 7b). Osteocalcin (OSC) demonstrated reduced expression at all time points measured (FIG. 7c). Interleukin-1 beta (IL1-b) mRNA expression progressively increased over the entire period with a positive modulation of approximately 2-fold by day 28 (FIG. 7d).

IL1-b has been shown to play a role in recruiting inflammatory cells, stimulating angiogenesis, enhancing extracellular matrix synthesis, and promoting the formation of the cartilaginous callus. IL1-b has been shown previously to have a biphasic expression pattern during bone healing, where within the first 24 hrs, macrophages express high levels of IL1-b, then expression declines to undetectable levels by day 3. The expression of IL1-b rises again approximately 3 weeks following the injury, mainly due to expression by osteoblasts. High concentrations of IL1-b stimulate proteases to degrade callus tissue and help with bone remodeling. The upregulation of the IL1-b expression within 28 days suggest the presence of the osteoblasts and perhaps bone remodeling. Further studies may evaluate the expression levels of Runx-2, ALP, and OSC at earlier time points including 3, 5, 7 and 10 days post implantation, since expression by these genes may have increased prior to day 14.

### 3.5 Evaluation of RNA-Seq Expression

To determine effector gene up/down-regulation in response to different treatments in ZDF rats, expression analyses of poly-A RNA extracted from explanted scaffolds were performed. A 4-fold up- or down-regulated change relative to Gel-CM control was defined as the threshold for determining differentially expressed genes after exposure to different treatments. The result showed large number of genes were down-regulated in the gene active matrices that are not down-regulated in the presence of INS MPs and/or VD3 with the control matrix. In addition, as shown in FIG. 4, the groups that were treated with ((INS MPs+VD3)Gel+GAM), ((VD3)Gel+GAM) or ((INS MPs)Gel+GAM) cluster more closely together in expression of the variable genes compared to the groups treated with GAM, ((INS MPs)Gel+CM) or (Gel+CM) in the absence of INS MPs and/or VD3. This data suggests that the presence of INS MPs and VD3 mitigates the downregulation of the genes compared to GAM alone. However, no statistical conclusion can be derived from these data set since the findings are based on research performed on n=1.

### 3.6. Assessment of In Vivo Bone Regeneration

The potential of the composite material to induce bone formation was investigated in vivo in a challenging diabetic rat model. The effect on ectopic bone formation following delivery of PEI-(pBMP-2+pFGF-2) nanoplexes, INS and VD3 after 28 days of implantation was studied in vivo quantitatively and qualitatively. High resolution micro-computed tomography (μCT) scanning was carried out to quantitatively and qualitatively assess the newly-formed bone tissue within the intramuscular (IM) pockets at 28 days post implantation.

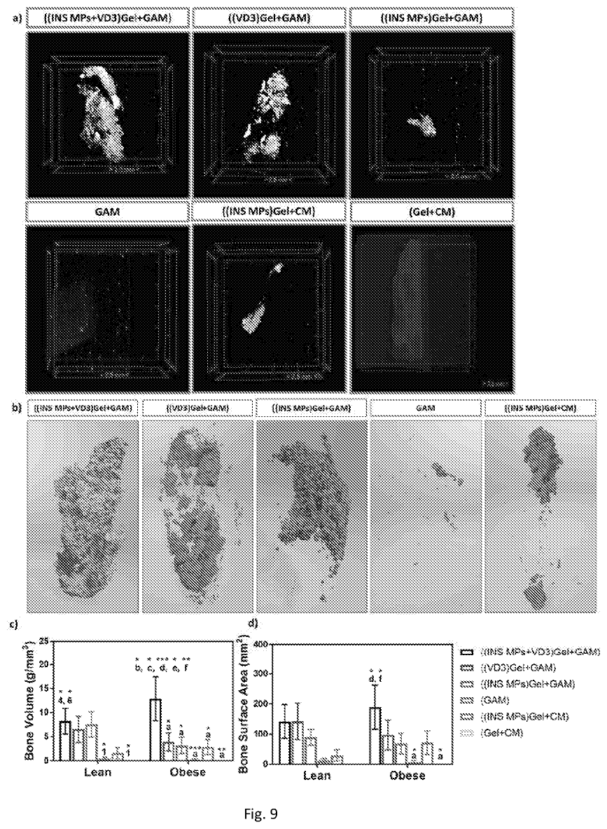

The μCT qualitative assessment of explanted constructs demonstrated an increase in mineralized tissue formation in the groups that were treated with ((INS MPs+VD3)Gel+GAM), ((VD3)Gel+GAM) or ((INS MPs)Gel+GAM) compared to the groups treated with GAM, ((INS MPs)Gel+CM) or (Gel+CM). The μCT images rarely showed newly induced bone in the groups treated with GAM or (Gel+CM) (FIG. 9a, 9b). This was confirmed by a quantitative analysis where the amount of de novo bone formation was assessed by analyzing the mineralized bone volume and bone surface area. In obese (ZDF) rats, there was significantly increased bone volume in the combinatorial ((INS MPs+VD3)Gel+GAM) group compared to all other treatment groups (FIG. 9c). Furthermore, the bone surface area showed the same trend that was observed with bone volume assessment. As shown in FIG. 5d in obese rats, the induced bone surface area was significantly higher in the group that was treated with ((INS MPs+VD3)Gel+GAM) compared to the GAM or (Gel+CM) group. In the lean (ZL) rats, relative to GAM and (Gel+CM) the presence of the INS MPs and VD3 together or individually in combination with GAM enhanced IM bone formation. There was significantly increased bone volume in the combinatorial ((INS MPs+VD3)Gel+GAM) group compared to GAM and (Gel+CM) treatment groups (FIG. 9c). A trend toward higher bone surface area of the induced bone was observed in the groups that received ((INS MPs+VD3)Gel+GAM), ((VD3)Gel+GAM) and ((INS MPs)Gel+GAM) treatments compared to the GAM or (Gel+CM) group, albeit not significantly (FIG. 9d). Also, the presence of plasmid loaded nanoplexes provided an enhancement in bone generation (1.5 fold increase) when compared to the collagen matrix alone (Gel+CM).

### 3.7. Histomorphometric Analysis

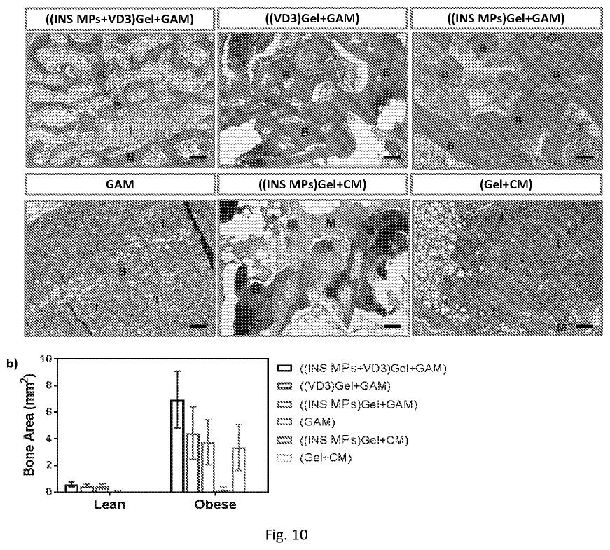

Finally, histological analyses of explanted constructs using hematoxylin and eosin (H&E) staining, confirmed the results from μCT measurements (FIG. 10). FIG. 6a, shows the increasing mineralization in the ((INS MPs+VD3)Gel+GAM), ((VD3)Gel+GAM), ((INS MPs)Gel+GAM), or ((INS MPs)Gel+CM) treated ZDF rats with no evidence of a local inflammatory response. And the formation of fibrosis in the group that was treated with GAM, or (Gel+CM). The control group failed to form new mineralized bone and mostly promoted soft tissue formation. Furthermore, this qualitative assessment was confirmed by quantifying the mineralized bone area via histo-photometric analysis. As shown in FIG. 10b in obese rats, the induced bone surface area was 1.6-1.9-fold higher in the group that was treated with ((INS MPs+VD3)Gel+GAM) compared to the group that received ((VD3)Gel+GAM), ((INS MPs)Gel+GAM), or ((INS MPs)Gel+CM) implants. In addition, the same trend was observed toward higher bone surface area (6-fold) of the induced bone in the groups that received ((INS MPs+VD3)Gel+GAM) compared the GAM or (Gel+CM) groups. In the lean (ZL) rats, histo-photometric analysis showed an enhancement in bone generation in the groups treated with ((INS MPs+VD3)Gel+GAM), ((VD3)Gel+GAM), or ((INS MPs)Gel+GAM), when compared to the ((INS MPs)Gel+CM), GAM, or collagen matrix alone (Gel+CM).

### CONCLUSION

In this study, a composite biomaterial containing pBMP-2, pFGF-2, INS and an active VD3 metabolite was tested for its capacity to induce bone formation in obese Type-2 diabetic rats. This implant formulation resulted in an effective bone generation response and promoted ectopic bone formation as evidenced by μCT analysis. The μCT analysis revealed that the mean bone volume (12.89 mm³ for ZDF and 8.24 mm³ for ZL) was significantly higher for the group treated with implants containing INS, VD3 and PEI-(pBMP-2+pPGF-2) nanoplexes. This suggests that the dose of INS. VD3 was in fact therapeutically advantageous when combined with PEI-(pBMP-2+pFGF-2) nanoplexes. This bone inductive composite biomaterial may be employed as a treatment for patients with fractures or reconstructive procedures such as arthrodesis or osteotomies, specifically diabetic patients, as they are prone to adverse fracture healing and increased mortality following fracture, or at risk from limb loss due to bony deformity in the feet. Further studies can assess bone regeneration capacity of the composite biomaterial in bone lesions.

### REFERENCES

Akinc et al., J. Gene Med., 7:657 (2005).
Atluri et al., Mol. Pharm., 12:3032 (2015).
Azad et al., J. Orthop. Trauma. 23:267 (2009).
Beam et al., J. Orthop. Res., 20:1210 (2002).
Bell & Polonsky, Nature, 414:788 (2001).
Boussif et al., Proc. Natl. Acad. Sci. U.S.A 92:7297 (1995).
Brown et al., PLoS One, 9:e99656 (2014).
Brownlee, Nature, 414:813 (2001).
Cortizo et al., Mol. Cell. Biochem. 250:1 (2003).
Elangovan et al., Biomaterials, 35:737 (2014).
Furst et al., J. Clin. Endocrinol. Metab., 101:2502 (2016).
Gandhi et al., Bone, 37:482 (2005).
Gerstenfeld et al., J. Cell. Biochem., 88:873 (2003).
Godbey et al., J. Control. Release. 60:149 (1999).
Gradinaru et al., Aging Clin. Exp. Res., 24:595 (2012).
Hamann et al., Am. J. Physiol. Endocrinol. Metab., 301:E1220 (2011).
Han et al., Life sciences, 55:948 (2012).
Heap et al., J. Pediatr., 144:56 (2004).
Hough et al., Endocrinology 108:2228 (1981).
Jianhong et al., Cell Biochem. Funct., 28:334 (2010).
Jiao et al., Current Osteoporosis Rep., 13:327 (2015).
Khorsand et al., J. Control. Release, 248:53 (2017).
Kon et al., J. Bone Miner. Res., 16:1004 (2001).
Manna et al., Arch. Biochem. Biophys., 615:22 (2017).
Maxwell & Wood, Nutr. Rev., 69:291 (2011).
Mountziaris & Mikos, Modulation of the Inflammatory Response for Enhanced Bone Tissue Regeneration, Tissue engineering. Part B. Reviews, 14:179 (2008).
Okazaki, Nihon Rinsho, 67:1003 (2009).
Paglia et al., J. Orthop. Res., 31:783 (2013).
Park et al., J. Orthop. Res., 331:776 (2013).
Parker et al., Maturitas, 65:225 (2010).
Plum & DeLuca. Nat. Rev. Drug. Discov., 9:941 (2010).
Pun et al., J. Bone Miner. Res., 4:853 (1989).
Santana et al., Diabetes, 52:1502 (2003).
Schmid et al., Dev. Dyn., 238:766 (2009).
Shively et al., J. Control. Release 33:237 (1995).
Sohn et al., Diabetes Care, 33:98 (2010).
SooHoo et al., J. Bone Joint Surg. Am., 91:1042 (2009).
Starup-Linde & Vestergaard, Bone, 82:69 (2016).
Takenaga et al., Int. J. Pharm., 271:85 (2004).
Thrailkill et al., Am. J. Physiol. Endocrinol. Metab., 289:E735 (2005).
Uchida et al., Pharm. Res., 11:1009 (1994).
Wagner et al., Proc. Natl. Acad. Sci. U.S.A, 88:4255 (1991).
Wang et al., Br. J. Oral Maxillofac. Surg., 49:225 (2011).
Wu et al., Bone, 52:1 (2013).
Xiong et al., Biochem. Biophys. Res. Commun., 494:626 (2017).
Zimmet et al., Nature, 4141:782 (2001).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A composition comprising:
a scaffold loaded with nucleic acid encoding at least two growth factors selected from BMP, FGF, IGF, HGF, PGF, PDGF, TGFB or VEGF;
a gel loaded with insulin and at least one form of bioavailable form of vitamin D;
wherein the scaffold encapsulates or surrounds at least a portion of the gel.

2. The composition of claim 1, wherein the nucleic acid is linear DNA, plasmid DNA, mRNA, cmRNA, or double-stranded RNA.

3. The composition of claim 1 or 2, wherein the scaffold comprises a natural polymer.

4. The composition of claim 3, wherein the natural polymer comprises collagen, proteoglycan, alginate, chitosan or extracellular matrix.

5. The composition of claim 1 or 2, wherein the scaffold comprises a synthetic polymer.

6. The composition of claim 5, wherein the synthetic polymer comprises PLA, PLGA, PLLA or polystyrene.

7. The composition of any one of claims 1 to 6, wherein the bioavailable form of vitamin D comprises calcitriol or ercalcitriol.

8. The composition of any one of claims 1 to 7, wherein the gel comprises fibrin.

9. The composition of any one of claims 1 to 8, further comprising insulin complexed with a first delivery vehicle.

10. The composition of claim 9, wherein the first delivery vehicle comprises microparticles or nanoparticles.

11. The composition of claim 9 or 10, wherein the first delivery vehicle comprises cationic or non-cationic polymers, cationic liposomes or cationic emulsions, or a synthetic polymer.

12. The composition of claim 9 or 10, wherein the first delivery vehicle comprises PEI, chitosan, cyclodextrin, dendrimers, PLGA, PLA, or PAMAM, alginate, polycaprolactone (PCL), or a polyanhydride.

13. The composition of any one of claims 1 to 12, wherein the plasmid DNA is complexed with a second delivery vehicle.

14. The composition of claim 13, wherein the second delivery vehicle comprises a synthetic polymer.

15. The composition of claim 14, wherein the synthetic polymer comprises PEI, PLGA, PLA, or PAMAM microparticles or nanoparticles.

16. The composition of any one of claims 1 to 15, wherein the gel controls the initial burst release of the insulin.

17. The composition of any one of claims 1 to 16, wherein the growth factor comprises BMP1, BMP2, BMP3, BMP4, BMP5, BMP6, BMP7, BMP8A, BMP8B, BMP10, BMP15, FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, IGF1, IGF2, HGF, NGF, PDGFA, PDGFB, PDGFC, PDGFD, TGFB1, TGFB4, TGFB3, VEGFA, VEGFB, VEGFC, or VEGFD.

18. The composition of any one of claims 1 to 17, wherein the nucleic acid is released from the composition before the bioavailable form of vitamin D.

19. The composition of any one of claims 1 to 18, wherein the bioavailable form of vitamin D is released from the composition before the insulin.

20. A method to treat bone injury in a mammal, comprising: administering the composition of any one of claims 1 to 19 to a mammal at a site of bone injury.

21. The method of claim 20 wherein the mammal is a human.

22. The method of claim 21, wherein the human is suspected to have diabetes mellitus.

23. The method of claim 20, 21 or 22, wherein the bone injury comprises at least one bone fracture.

24. The method of any one of claims 20 to 23 wherein the composition comprises a collagen scaffold.

25. The method of any one of claims 20 to 24 wherein the composition comprises a fibrin gel.

26. The method of any one of claims 20 to 25 wherein the microparticles or nanoparticles comprise the insulin.

27. The method of claim 26 wherein the microparticles or nanoparticles are formed of lactic acid, glycolic acid, or combinations thereof.
